(19) United States
(12) Patent Application Publication
(10) Pub. No.: US 2017/0356895 A1
(43) Pub. Date: Dec. 14, 2017

(54) TOXICANT ASSAYS FOR CONSUMABLE PRODUCTS

(71) Applicant: VITARGENT (INTERNATIONAL) BIOTECHNOLOGY LIMITED, SHATIN (HK)

(72) Inventors: XUEPING CHEN, SHATIN (HK); ZIXIANG CHEN, SHATIN (HK); WAI LEUNG TAO, SHATIN (HK)

(21) Appl. No.: 15/600,438

(22) Filed: May 19, 2017

Related U.S. Application Data
(60) Provisional application No. 62/347,129, filed on Jun. 8, 2016.

(51) Int. Cl. G01N 33/02; A61K 49/00

(52) U.S. Cl. CPC G01N 33/02; A61K 49/0008; A01K 2267/03; G01N 2333/4603

(57) ABSTRACT

Methods of determining whether a toxicant is present in a consumable product, which comprise contacting a teleost embryo with an extract from a sample of the consumable product and determining whether the extract exerts a toxicity effect on the embryo.

### 1. FIELD OF INVENTION

The invention relates to methods for determining whether a toxicant is present in a consumable food. Particularly, the invention relates to methods of using teleost embryo in assays to determine the presence of a toxicant in a consumable food.

### 2. BACKGROUND

With the rise in modernization and globalization, consumable products, such as foods and beverages, can go through many processes in which potentially toxic chemicals can enter the products before reaching the consumer. The US Environmental Protection Agency tracks or regulates more than 100,000 chemicals (Substance Registry Services Fact Sheet, available at ofmpub.epa.gov/sor_internet/registry/substreg/educationalresources), and the toxicity of many of these chemicals has not been well studied, especially their total biological toxicity when combined with other chemicals. Ensuring the safety of consumable products is a great challenge to the modern testing industry given the sheer number of potentially toxic chemicals and chemical combinations that can find their way into consumable products.

To date, toxicity testing of consumables still largely relies on chemical-specific tests, especially chemical analysis. For example, a review article introduces determination of pesticide residues in food matrices using QuEChERs methodology (Angelika Wilkowsk and Marek Biziuk, Food Chemistry 125 (2011), pp. 803-812). While chemical-specific tests can be sensitive and precise, they can fail to detect unknown toxicants that are not intended to be specifically tested; this can allow unanticipated toxicants to go undetected. Even in cases where the chemical composition of a sample is known in detail, its effective toxicity cannot necessarily be reliably predicted due to the lack of knowledge concerning the effects of chemical mixtures. Practical experience with studies has shown that chemical-specific measurements identify true toxicity in unknown samples only about 20% of the time, which means up to 80% toxicants are unidentified.

Thus, new methods for determining whether a toxicant is present in a consumable product are needed. US 2013/152222 relates to transgenic fishes and their use in, inter alia, detecting estrogenic and anti-estrogenic compounds, monitoring estrogen-like activity in the environment, and elucidating liver regeneration. However, there is a need to develop a bioassay to detect toxicants in a sample.

### 3. SUMMARY

The disclosure provides methods of determining whether a toxicant is present in a consumable product, such as a food or beverage, which comprise contacting a teleost embryo with an extract from a sample of a consumable product and determining whether the extract exerts a toxicity effect on the embryo, where a toxicity effect on the embryo is indicative of the presence of a toxicant in the consumable product. Exemplary consumable products that can be tested using the methods of the disclosure are described in Section 4.2. In some embodiments, the extract comprises an organic solvent extract (e.g., an acetonitrile extract), which is optionally dehydrated and/or delipidated. In one embodiment, the delipidation is only for a sample that contains lipid or rich-lipid. For non or low lipid containing samples, no delipidation is needed. Methods for preparing an extract from a consumable product for toxicant testing are described in Section 4.4 and in numbered embodiments 99 to 108 below.

In some embodiments, the testing methods of the disclosure comprise determining whether the extract exerts a toxicity effect on teleost embryos, such as an acute effect (e.g., malformation or death) or a specific effect (e.g., estrogen activity disruption). In some embodiments, the testing methods comprise contacting a teleost embryo with an organic solvent extract that is obtainable or obtained by a process as described in Section 4.3 or one of numbered embodiments 14 to 43, 55 to 58 or 99 to 108 below. Exemplary methods of determining whether a toxicant is present in a consumable product are described in Sections 4.4 and numbered embodiments 1 to 99 below. In some embodiments, the teleost is a medaka or a zebrafish embryo (as described in Sections 4.4.1-4.4.2), and in some embodiments can be transgenic. Acute and specific toxicity effects that can be determined using the methods of the disclosure are described in Sections 4.4.3 and 4.4.4, respectively. Exemplary acute toxicity effects include mortality, and malformation, and exemplary specific toxicity effects include estrogen activity disruption, androgen activity disruption, xenobiotic effect, cardiotoxicity effect, and hepatotoxicity effect.

The sample testing methods provided by the disclosure can be used, for example, to evaluate the total biological toxicity effects of extracts from consumable products. The methods of the disclosure can be applied in a high throughput manner to test large numbers of samples, providing, for example, a means to determine the biological safety of a large number of consumable products.

### 4. DETAILED DESCRIPTION

### 4.1. Overview

The invention creates a biological assay method for determining a toxicity profile in a consumable product sample. Significantly different from a chemical assay directed to detection of a specific toxicant(s) but not unspecified toxicants, the biological assay of the invention obtains an overall toxicity profile in the sample that can be used as index of toxicity of a sample. In contrast to the chemical assay, no toxic substance is added to the sample during the sample pretreatment process used in the method of the invention and agents used in the method will not react with the sample and change sample toxicity.

The disclosure provides methods of determining whether a toxicant is present in a consumable product. The methods comprise contacting a teleost embryo with an extract from a sample of the consumable product and determining whether the extract exerts a toxicity effect on the embryo, where a toxicity effect on the embryo indicates the presence of a toxicant in the consumable product. Consumable products which can be tested using the methods of the disclosure include, but are not limited to, feed, human foods, pet foods, and beverages. Advantageously, the methods of the disclosure can be used to monitor the total biological toxicity of a consumable, in contrast to chemical specific tests which generally detect the presence of only one type of toxicant or one group of toxicants. Methods of determining overall toxicity in a consumable product are illustrated in Section 4.2. Exemplary consumable products that can be tested using the methods of the disclosure are described in Section 4.3. Processes for preparing extracts from samples of consumable products are described in detail in Section 4.4, and methods of determining whether a toxicant is present in the consumable product extract are described in Section 4.5.

### 4.2. Method of Determining Overall Toxicity in a Consumable Product

In one aspect, the invention provides a method of determining an overall toxicity in a consumable product, comprising:
a) combining a polar organic solvent and the consumable product to obtain a polar organic extract with an overall toxicity;
b) contacting a teleost embryo with the organic extract of a);
c) determining whether the extract exerts a toxicity effect on the embryo;
   wherein a toxicity effect on the embryo shows an overall toxicity of the consumable product.

The polar organic solvent is used to extract most toxicants in a consumable product. Examples of the polar organic solvent include, but are not limited to, acetonitrile, methanol, ethanol, propanol, isopropanol, acetone, ethyl acetate, propanol, isopropanol, and a mixture containing two or more solvents thereof. In one embodiment, the volume of organic solvent combined with the sample to form the mixture can be, for example, 1 to about 5 times the volume or weight of the sample. That is, when the sample is a liquid, the volume of the polar organic solvent is about 1 to about 5 times the volume of the sample; when the sample is a solid or semi-solid, the volume of the polar organic solvent is about 1 to about 5 times the weight of the sample. Preferably, the volume of organic solvent combined with the sample to form the mixture can be about 1 to about 4 times, about 1 to about 3 times, about 1 to about 2 times or about 1 times the volume or weight of the sample.

In one embodiment, before the above step a), water is added to the consumable product when the product is water unsaturated.

In another aspect, the invention provides a method of determining an overall toxicity in a lipid-containing consumable product, wherein the extract of step a) is further delipidated.

In one embodiment, the lipid-containing consumable product contains a lipid that causes the polar solvent extract cannot be completely dried. Preferably, the lipid is in an amount of higher than 5% (w/w). More preferably, the lipid is in an amount of higher than 10% (w/w), 20% (w/w), 30% (w/w), 40% (w/w), 50% (w/w), 60% (w/w), 70% (w/w), 80% (w/w) or 90% (w/w). In one embodiment, the delipidation step is performed by adding a non-polar solvent to the organic solvent extract. Examples of the non-polar solvent include, but are not limited to, pentane, cyclopentane, hexane, cyclohexane, benzene, toluene, chloroform, diethyl ether, dichloromethane and a mixture containing two or more solvents thereof.

In one embodiment, the method of the invention can be used to determine an overall toxicity in an edible oil such as vegetable and nut oils, as well as lard and other animal fats. In one embodiment, zebrafish embryos are used for toxicity testing, which offers an accurate and quick approach to assess the safety of edible oils. For example, a testing method, combining a simple and easy edible oil extraction method, with a zebrafish embryo-based acute toxicity test to measure the acute toxicity (such as LC50) of the extract is established.

In one embodiment, the chemical contaminants of the consumable product are determined by a chemical analysis method to establish the correlation between the teleost embryo toxicity test and chemical contaminants.

### 4.3. Consumable Products

The consumable product can be a product that is intended for human consumption (e.g., a food or a beverage) or animal consumption (e.g., pet foods such as a dog food or a cat food or livestock feed such as pig, goat or chicken feed). In some embodiments, the consumable product is intended for human consumption. In other embodiments, the consumable product is intended for animal consumption. Consumable products that can be tested using the methods of the disclosure include foods, beverages, and ingredients used to make a food or a beverage. As used herein, the term “food” encompasses food for human consumption and feed for animal consumption (including for consumption by livestock and for consumption by pets). Foods that can be tested include edible oil, ready to eat foods (e.g., cooked foods, canned foods, foods packaged in single or multiple serving packages, or animal feed), dairy products, meats, vegetables, fruits, infant formula, and dietary supplements derived from dairy, meat, vegetables, fruits, or a combination thereof (e.g., protein powders made from dairy, meat, or vegetables). As used herein, the term “edible oil” refers to a food substance, other than a dairy product, of whatever origin, source or composition that is manufactured for human consumption wholly or in part from a fat or oil other than that of milk Edible oils that can be tested include plant-derived edible oils or fats, animal-derived edible oils or fats and synthetic oils and fats.

Exemplary dairy products that can be tested using the methods of the disclosure include milk (e.g., fresh milk, condensed milk, or powdered milk), buttermilk, cream, ice cream, yogurt, butter, cheese, and protein powders (e.g., whey concentrate, whey isolate, casein concentrate, or casein isolate).

Exemplary meats that can be tested using the methods of the disclosure include beef, pork (e.g., ham), lamb, goat, seafood (e.g., fish, shrimp, lobster, crab, clams, oysters, octopus, or squid), and poultry (e.g., chicken, duck, turkey, or goose). Meat can be fresh, cooked, or processed (e.g., dried or salted).

Exemplary fruits that can be tested using the methods of the disclosure include bananas, mangos, citrus fruits (e.g., oranges, lemons, limes, or grapefruit), apples, pears, peaches, plums, pineapples, berries (e.g., strawberries, blackberries, raspberries, or cranberries), lychees, and grapes. Exemplary vegetables include cabbages, turnips, radishes, carrots, lettuces, beans, peas, potatoes, eggplants, squashes, and onions. Fruits and vegetables can be fresh, cooked, or processed (e.g., jellies, jams, potato chips).

Exemplary beverages that can be tested using the methods of the disclosure include soft drinks (e.g., beverages containing milk, tea, coffee, juice, or sugar, or a combination thereof), and alcoholic drinks (e.g., beer, wine, cider, or spirits).

Exemplary food ingredients that can be tested using the methods of the disclosure include table sugar, brown sugar, corn syrup, carboxymethylcellulose, maltodextrin, demineralized whey powder, lactose, and oligosaccharides.

Exemplary edible oils that can be tested using the methods of the disclosure include olive oil, palm oil, soybean oil, canola oil (rapeseed oil), corn oil, peanut oil, corn oil, cottonseed oil, rice bran oil, coconut oil, peanut oil, sesame oil, pumpkin oil, sunflower oil, walnut oil, mustard oil, other vegetable oils and fats, butter, lard, beef tallow, other animal-based oils and fats and margarine.

The foregoing exemplary categories are not intended to be limiting, and inclusion of a consumable product in one category does not exclude its inclusion in another. For example, milk can be considered a dairy product and a beverage.

### 4.4. Consumable Product Extracts

Extracts that can be used in the toxicity testing methods of the disclosure are prepared from samples of consumable products, such as those described previously in Section 4.3. The sample can be an entire product (e.g., the entire contents of a single serving package of food) or a portion thereof. The sample can be, but is not necessarily, homogenized prior to extraction. Methods for homogenizing samples are known in the art, and include grinding (e.g., using a mortar and pestle), blending (e.g., with a blender), and sonication. Extract preparation from some consumable products may not benefit from a homogenization step, such as homogeneous dairy products or beverages, while others, such as meats or ready-to-eat foods such as rice dishes may benefit from a homogenization step prior to extraction.

Extracts that can be used in the toxicity testing methods of the disclosure are preferably polar organic solvent extracts. The term “polar organic solvent” when used in connection with the term “polar organic solvent extract” refers to the particular organic solvent or mixture of polar organic solvents used to extract compounds from a sample of a consumable product and does not necessarily refer to the solvent in which the extract may be dissolved in at any given time. For example, an extract prepared by extracting compounds from a sample using acetonitrile remains an acetonitrile extract even in instances in which the extract is processed to remove the acetonitrile following extraction. Thus, for example, an acetonitrile extract that has been dried to remove the acetonitrile and subsequently redissolved or resuspended in another solvent (such as methanol) remains an acetonitrile extract even though the extract in its present state contains another solvent other than acetonitrile.

Exemplary organic solvent extracts include acetonitrile extracts, methanol extracts, ethanol extracts, propanol extracts, isopropanol extracts, acetone extracts, ethyl acetate extracts, propanol extracts, isopropanol extracts, and a mixture containing two or more solvents thereof. In some embodiments, the organic solvent extract is an acetonitrile extract. In other embodiments, the polar organic solvent extract is a methanol extract or an ethanol extract. Processes for making organic solvent extracts and reagents that can be used to make polar organic solvent extracts are described in Section 4.4.1.

Polar organic solvent extracts can optionally be dehydrated and/or can optionally be delipidated. Processes for dehydrating an organic solvent extract and reagents that can be used to dehydrate an organic solvent extract are described in Section 4.4.2. Processes for delipidating an organic solvent extract and reagents the can be used to delipidate an organic solvent extract are described in Section 4.4.3.

4.4.1. Extract Preparation

Polar organic solvent extracts can be obtained by a process in which the first step comprises forming a mixture comprising a sample of the consumable product (e.g., a homogenized sample), a polar organic solvent and, optionally, a first salt and/or a sugar. In some embodiments, the mixture comprises the sample, the polar organic solvent, and a first salt. In other embodiments, the mixture comprises the sample, the polar organic solvent and a sugar. In other embodiments, the mixture comprises the sample, the polar organic solvent, a first salt and a sugar.

Without being bound by theory, it is believed that the first salt and/or sugar can promote the formation of at least two liquid phases in the mixture, one of which is enriched in the organic solvent relative to the other phase(s), and can promote the extraction of toxicants from the mixture into the phase enriched in the polar organic solvent. For example, acetonitrile is water miscible, and the addition of a salt or a sugar to a mixture containing acetonitrile and water can help to establish two liquid phases, one of which is enriched in acetonitrile. When using a polar organic solvent that is immiscible with a liquid contained in the sample (e.g., when forming a mixture of a water immiscible organic solvent such as toluene and a water containing food sample), the addition of salt may not be necessary to establish two liquid phases, although use of a first salt and/or sugar may aid in the extraction of toxicants from the mixture to the polar organic solvent.

The volume of the polar organic solvent can be selected or varied based upon the amount and/or nature of the sample (e.g., the consistency of the sample). The volume of polar organic solvent combined with the sample to form the mixture can be, for example, about 1 to about 5 times the volume or weight of the sample; that is, when the sample is a liquid, the volume of the polar organic solvent is about 1 to about 5 times the volume of the sample; when the sample is a solid or semi-solid, the volume of the polar organic solvent is about 1 to about 5 times the weight of the sample. (e.g., about 1 to about 5 times, about 1 to about 4 times, about 1 to about 3 times, about 1 to about 2 times), about 1.5 to about 5 times the volume or weight of the sample (e.g., about 1.5 to about 4 times, about 1.5 times to about 3 times), about 2 times to about 5 times (e.g., about 2 to about 5 times, about 2 to about 4 times, about 2 times to about 3 times), or about 2 times to about 4 times the volume or weight of the sample). In some embodiments, the volume of the polar organic solvent (e.g., acetonitrile) is at least 1.5 times the volume or weight of the sample. Higher polar organic solvent to sample ratios can in some instances allow for higher amounts of extracted toxicants in contrast to lower polar organic solvent to sample ratios; however, higher polar organic solvent to sample ratios require more reagents (e.g., solvents and salts) and result in more dilute extracts.

First salts that can be used in the extraction process include sodium chloride, magnesium sulfate, sodium sulfate, calcium sulfate, calcium chloride, magnesium chloride, sodium acetate, ammonium acetate, anhydrous sodium sulfate, anhydrous magnesium sulfate, anhydrous sodium sulfate, anhydrous calcium sulfate, anhydrous calcium chloride, anhydrous calcium sulfate, and combinations thereof. The use of an anhydrous salt can help to saturate water solubility and “squeeze” the toxicants into organic solvent layer. In the context of this disclosure, and unless required otherwise by context, a salt that is not specifically identified as being in hydrated or anhydrous form encompasses both hydrated and anhydrous forms of the salt. For example, “calcium chloride” encompasses anhydrous and hydrated forms of calcium chloride (i.e., CaCl₂(H₂O)ₓ, where x=0, 1, 2, 4, or 6).

In some embodiments, the first salt comprises sodium chloride, magnesium sulfate, sodium sulfate, calcium sulfate, calcium chloride, magnesium chloride, sodium acetate, ammonium acetate, or a combination thereof. In some embodiments, the salt comprises sodium chloride.

In other embodiments, the first salt comprises a combination of (i) sodium chloride, magnesium sulfate, calcium chloride, magnesium chloride, sodium acetate, ammonium acetate, or a combination thereof, and (ii) anhydrous sodium sulfate, anhydrous magnesium sulfate, anhydrous sodium sulfate, anhydrous calcium sulfate, anhydrous calcium chloride, anhydrous calcium sulfate, or a combination thereof. For example, the first salt can be a combination of sodium chloride and anhydrous sodium sulfate.

Sugars that can be used in the extraction process include monosaccharides and disaccharides, such as glucose, xylose, arabinose, fructose, maltose, sucrose and mixtures thereof. In some embodiments, the sugar comprises sucrose.

The first salt and/or sugar can be combined with the sample of the consumable product and the polar organic solvent to form the mixture, or the first salt and/or sugar can be added to a preformed mixture comprising the sample and the polar organic solvent. When the first salt and/or sugar is added to a preformed mixture, the mixture can be mixed before and/or after combining the mixture with the salt and/or sugar (e.g., by shaking the mixture, vortexing the mixture, sonicating the mixture, or vortexing and sonicating the mixture). The first salt and/or sugar can be added to the preformed mixture until the mixture is saturated with the first salt and/or sugar (e.g., as indicated by the observance of salt or sugar crystals within the mixture that do not dissolve).

Following formation of the mixture comprising the sample, the polar organic solvent and, optionally, a first salt and/or a sugar, the polar organic solvent extract can be obtained from the mixture by separating a phase containing the organic solvent from the mixture (i.e., a phase enriched in the organic solvent relative to the other phase(s)). Separation can comprise centrifuging the mixture to separate the phase containing the polar organic solvent from the mixture. Alternatively, the phases can be separated under the force of gravity, although separating a mixture under the force of gravity may take longer to complete compared to separating the mixture using centrifugation. In some embodiments, the mixture can be mixed prior to the separation (e.g., by shaking the mixture, vortexing the mixture, sonicating the mixture, or vortexing and sonicating the mixture). Without being bound by theory, it is believed that mixing the mixture prior to separation can increase the yield of extracted toxicants.

Following separation, the polar organic solvent extract can be recovered, for example, by pipetting the phase containing the polar organic solvent away from the other phases, decanting the separated phases, or separating the phases using a separatory funnel (e.g., when centrifugation is not used to separate the phases). The recovered polar organic solvent extract can be further processed, for example, to remove water (e.g., as described in Section 4.3.2), to remove lipid (e.g., as described below in Section 4.3.3), to remove the polar organic solvent used for extraction, or any combination thereof. Further processing such as delipidation in some embodiments is not performed. For example, in some embodiments, delipidation steps are not performed on polar organic solvent extracts made from samples comprising no lipids or low amounts of lipids.

The polar organic solvent used for extraction can be partially or completely removed by partially or completely by drying the polar organic solvent extract, for example under a stream of nitrogen or using a rotary evaporator. Alternatively, the polar organic solvent used for extraction can be removed by performing a solvent extraction on the organic solvent extract with another solvent. For example, when a solvent is used as the organic solvent for extracting compounds from the sample of the consumable product (e.g., a lipid rich product), a polar organic solvent (e.g., acetonitrile, dimethylformamide, or dimethyl sulfoxide) can be used to extract compounds (e.g., toxicants) and then hexane can be used to extract lipid from the polar organic extract. The polar organic solvent can then be removed from the extract if desired, for example by drying the extract under a stream of nitrogen or using a rotary evaporator.

Polar organic solvent extracts that have been dried can be redissolved or suspended in a second organic solvent. An organic solvent used for extraction can be removed at any time following extraction, for example, before or after subjecting the organic solvent extract to dehydration, or before or after subjecting the organic solvent extract to delipidation. In some embodiments, the organic solvent used for extraction is removed following dehydration and delipidation. In some embodiments, the second organic solvent is a solvent that is appropriate for use in a toxicity assay as described in Section 4.4.4. Exemplary second organic solvents include methanol, dimethyl sulfoxide, and mixtures thereof.

4.4.2. Dehydration

A polar organic solvent extract, for example prepared by a process as described in Section 4.4.1, can be dehydrated to remove water that may be present in the polar organic solvent extract. For example, an acetonitrile extract prepared from a liquid containing food product can contain residual water. Polar organic solvent extracts containing water in addition to a polar organic solvent can be dehydrated by combining the extract with a second salt to form a mixture, and then separating a phase containing the organic solvent from the mixture. The second salt can be added to the polar organic solvent extract until the mixture is saturated in the second salt (e.g., as indicated by the presence of salt crystals on the surface of the mixture or within the mixture). After formation of the mixture, the mixture can be mixed (e.g., by shaking the mixture, vortexing the mixture, sonicating the mixture, or vortexing and sonicating the mixture).

The second salt can be an anhydrous salt, such as anhydrous sodium sulfate, anhydrous magnesium sulfate, anhydrous calcium sulfate, anhydrous calcium chloride, or a combination thereof. Other salts that can absorb water can also be used. In some embodiments, the second salt comprises anhydrous sodium sulfate.

Following formation of the mixture comprising the second salt and the polar organic solvent extract, a phase containing the polar organic solvent can be separated from the mixture. Separation can comprise centrifuging the mixture to separate the phase containing the polar organic solvent from the mixture. Alternatively, the phases can be separated under the force of gravity. Following separation, the polar organic solvent extract can be recovered, for example, by pipetting the phase containing the polar organic solvent away from the other phases, decanting the separated phases, or separating the phases using a separatory funnel (e.g., when centrifugation is not used to separate the phases).

4.4.3. Delipidation

A polar organic solvent extract, for example prepared by a process as described in Section 4.3.1 or 4.3.2, can be delipidated to remove lipids that may be present in the polar organic solvent extract. Removal of lipids can be accomplished, for example, by washing the polar organic solvent extract at least once (e.g., once, twice, or three times) with a non-polar solvent. For example, C₅-C₈ alkanes (e.g., n-pentane, n-hexane, n-heptane, or n-octane) can be used. In some embodiments, the solvent used for delipidation comprises hexane. As used herein, “hexane” refers to n-hexane.

Delipidation can be performed on a polar organic solvent extract prepared as described in Section 4.4.1 or 4.4.2 without any intervening processing steps. Delipidation can also be performed on a polar organic solvent extract prepared as described in Section 4.4.1 or 4.4.2 that has undergone further processing steps, for example, partial solvent removal. The volume of non-polar solvent (e.g., hexane) used in each wash can be, for example, approximately one half to two thirds of the volume of the organic solvent extract (e.g., about 50%, about 55%, about 60%, or about 65%).

As an alternative to performing a polar organic solvent extraction followed by a wash with a nonpolar solvent as described above, a delipidated organic solvent extract can be obtained by using hexane as the solvent to extract compounds from the consumable product and then subjecting the hexane extract to solvent extraction using a polar organic solvent such as acetonitrile as described above in Section 4.3.1. This embodiment can be used, for example, to make delipidated extracts from lipid rich samples.

Delipidation is only for a sample that contains lipid or lipid rich. For non or low lipid containing samples, no delipidation is needed. It has been found that if a delipidation step is not performed on lipid containing samples, the organic extract cannot be completely dried, resulting to varying final volume between samples, even when processed identically. Thus, delipidation of lipid containing samples can help to standardize the volume of organic solvent extract obtained when processing multiple samples. For samples containing no lipid or low amounts of lipid (e.g., low fat or fat free fruits and vegetables), it may be desirable to omit a delipidation step.

4.4.4. Exemplary Extraction Protocol

The following protocol is an exemplary protocol for preparing a dehydrated and delipidated acetonitrile extract from a consumable product such as a food.
1) combine a sample of the consumable product with an amount of acetonitrile to form a mixture, and optionally mix the mixture;
2) add sodium chloride to the mixture until saturation, and optionally mix the mixture;
3) separate a phase containing acetonitrile from the mixture (e.g., by centrifugation) to obtain an acetonitrile extract;
4) add anhydrous sodium sulfate to the acetonitrile extract until saturation to form a second mixture, and optionally mix the second mixture;
5) separate a phase containing acetonitrile from the second mixture (e.g., by centrifugation) to obtain a dehydrated acetonitrile extract;
6) wash the dehydrated acetonitrile extract with hexane, and optionally repeat once to obtain a dehydrated and delipidated acetonitrile extract; and
7) optionally remove the acetonitrile from the dehydrated and delipidated acetonitrile extract and redissolve the dehydrated and delipidated acetonitrile extract in methanol.

The following protocol is an exemplary protocol for obtaining a dehydrated and delipidated hexane extract from a consumable product such as a lipid rich food.
1) combine a sample of the consumable product with an amount of hexane to form a mixture, and optionally mix the mixture;
2) add sodium chloride to the mixture until saturation, and optionally mix the mixture;
3) separate a phase containing hexane from the mixture (e.g., by centrifugation) to obtain a hexane extract;
4) add anhydrous sodium sulfate to the hexane extract until saturation to form a second mixture, and optionally mix the second mixture;
5) separate a phase containing hexane from the second mixture (e.g., by centrifugation) to obtain a dehydrated hexane extract;
6) perform a solvent extraction on the dehydrated hexane extract using acetonitrile (keeping the acetonitrile layer) to obtain a dehydrated and delipidated hexane extract; and
7) optionally remove the acetonitrile from the dehydrated and delipidated hexane extract and redissolve the dehydrated and delipidated hexane extract in methanol.

The organic solvent extract produced using the foregoing exemplary extraction protocol is referred to as a hexane extract throughout the protocol even though the protocol includes a second solvent extraction step using acetonitrile because hexane is the solvent used to perform the initial solvent extraction on the sample of the consumable product (see Section 4.4).

### 4.5. Testing for a Toxicity Effect

Teleost embryos are an effective in vivo model system to screen/identify the biological effects, e.g., toxicity effects, of a test sample, and the adverse effects identified using fish (e.g., zebrafish and medaka fish) embryos is predictable to that of human beings. Fish embryos are not defined as protected animals under European legislation can be used as animal alternatives (Directive 2010/63/EU; Halder et al., 2010, Integrated Environmental Assessment Management. 6:484-491).

The screening assays of the disclosure entail contacting a teleost embryo with an extract from a sample of the consumable product and determining whether the extract exerts a toxicity effect on the embryo.

The teleost embryos that can be used in a screening assay of the disclosure can be of various freshwater, brackish water, or saltwater (marine water) species of fish, including, without limitation, fish of the Oryzias genus, the Danio genus and the Pimephales genus. Fish in the Oryzias genus belong to the Adrianichthyidae family and include, for example, Oryzias melastigma (alternative name Oryzias dancena) (marine or brackish medaka), Oryzias latipes (Japanese medaka), Oryzias celebensis, Oryzias marmoratus, Oryzias matanensis, Oryzias nigrimas (black buntingi), Oryzias orthognathus (buntingi), and Oryzias profundicola. Fish in the Danio genus belong to the Cyprinidae family and include, for example, Danio rerio (zebrafish), Danio albolineatus, Danio abolineatus, Danio choprae, Danio dangila, Danio erythromicron, Danio feegradei, Danio kerri, Danio kyathit, Danio margaritatus, Danio meghalayensis, Danio nigrofasciatus, and Danio roseus. Fish in the Pimephales genus belong to the Cyprinidae family and include Pimephales notatus (bluntnose minnow), Pimephales promelas (fathead minnow), Pimephales tenellus (slim minnow), and Pimephales vigilax (bullhead minnow). In particular embodiments, the fish embryos are Japanese or brackish medaka fish, zebrafish or fathead minnow embryos. Particular advantages of brackish medaka fish and zebrafish are described in Sections 4.5.1 and 4.5.2, respectively.

The toxicity effect can be an acute toxicity effect (as described in Section 4.5.3) or a specific toxicity effect (as described in Section 4.5.4).

The fish embryos can be transgenic or non-transgenic. Non-transgenic fish can be used, for example, for detection of an acute toxicity effect in extracts from consumable products, e.g., toxicity, as described in Section 4.5.3. Transgenic fish embryos are particularly useful when screening for a specific effect, e.g., for detection of estrogenic compounds and anti-estrogenic compounds in extracts from consumable products as described in Section 4.5.4.1 below.

The screening assays can be performed in a high throughput or semi high throughput manner, e.g., in multiwell plates (e.g., 24, 96 or 384 well plates), and/or with positive and/or negative controls (e.g., medium only as a negative control and an agent known to exert a toxicity effect in the particular assay as a positive control). Each extract in an assay can be tested in duplicate or triplicate. The assays can be performed using multiple dilutions of each extract.

4.5.1. Medaka Fish

The brackish medaka fish (Oryzias melastigma) is native to coastal waters and fresh waters in Pakistan, India, Burma and Thailand (Naruse, 1996, Fish Biol. L. Medaka 8:1-9), and thrives in waters of varying salinity ranging from 0 parts per thousand (ppt) to as high as 35 ppt. Additionally, this brackish medaka fish has a number advantages for transgenic development, including: (1) small size (2-3 cm for adult fish); (2) relatively short generation time (2-3 months); (3) dimorphic sex (e.g., females have a flat distal surface of the anal fin, while that of males is convex due to separated longer fin rays); (4) high prolific capacity to reproduce; (5) translucent eggs and larvae (up to 15 days post fertilization), which facilitates the positioning of DNA microinjection needles and observation of internal organs; and (6) adaptable to various transgenic techniques used to produce transgenic fish of other Oryzias species (e.g., Oryzias latipes).

Regarding the highly prolific capacity of the brackish medaka fish to reproduce, spawning of this fish can be induced all year round, and each pair of female and male fish can produce 20-30 eggs daily for up to several months under indoor maintained conditions (e.g., 28±1° C. with a constant light cycle of 14 h-light/8 h-dark and fed with commercial hormone-free flake food and brine shrimp (Artemia salina)). Eggs usually hatch in 11 to 15 days at 28±1° C.

The two medaka species of Oryzias melastigma and Oryzias latipes share high morphological, physiology, and genomic similarity, and while Oryzias latipes was first used to produce transgenic fish, the transgenic techniques were readily adapted to the brackish medaka Oryzias melastigma (Chen et al., 2008, Ectoxicol. Environ. Saf 71:200-208; Chen et al., 2009, Comp. Biochem. Physiol. C. Toxicol. Pharmacol. 149:647-655).

Medaka can be bred to be see-through (see, e.g., U.S. Pat. No. 6,737,559), further facilitating screening assays, particularly those involving detecting reporter expression or activity levels.

4.5.2. Zebrafish

Research has shown that zebrafish are a good model to predict toxicity of human drugs. There are close physiological and genetic similarities between zebrafish and mammalian species, and researchers have conducted systematic evaluations of zebrafish toxicity end points using large numbers of pharmacologically relevant compounds.

As an experimental tool, zebrafish have an array of advantages such as optical transparency, high fecundity, and quick, external development. Changes to morphology and modulations in gene and protein expression can be easily assayed through the use of fluorescent proteins. The relatively small physical size allows for multiple zebrafish to fit into a multiwell plate, making the scaling of experiments an easy transition. Also, the relatively cheaper costs associated with fish husbandry, coupled with the frequency of progeny that zebrafish can achieve, are other reasons that make this organism an attractive tool for screening assays.

4.5.3. Acute Toxicity Effect

The consumable product extracts of the disclosure can be measured for acute toxicity effects such as mortality and malformation on a whole organism level.

Taking zebrafish as an example, the zebrafish embryo toxicity test is based on a 48 h exposure of newly fertilized eggs in a static or semi-static system. Various endpoints such as coagulation of eggs and embryos, failure to develop somites, lack of heart-beat as well as non-detachment of the tail from the yolk are indicative of toxicity. These endpoints can be recorded after, e.g., 24, 48, 72 and 96 hr and used for the calculation of an LC₅₀ value of a consumable product extract. Analogous endpoints can be measured in Japanese medaka fish and in fathead minnows (see Braunbeck & Lammer, 2006, Background Paper on Fish Embryo Toxicity Assays, available from www.oecd.org/chemicalsafety/testing/36817242.pdf).

4.5.4. Specific Toxicity Effect

The consumable product extracts of the disclosure can also be assayed for specific effects, i.e., effects on particular tissue, organ, or hormone system. Assays of particular interest include those for cardiotoxicity, ototoxicity, seizure liability, endocrine disruption, gastrointestinal motility, hepatotoxicity, skin pigmentation alterations, muscle toxicity, pancreatic toxicity, carcinogenesis, neurotoxicity, and renal toxicity (see, e.g., Sarvaiya et al., 2014, Veterinary Clinical Science 2(3):31-38, Peterson and MacRae, 2011, Annu. Rev. Pharmacol. Toxicol. 52:433-53, Eimon and Rubenstein, 2009, Expert Opin. Drug Metab. Toxicol. 5(4):393-401, and references cited therein for assay details).

In certain aspects, specific toxicity effect can be measured by detecting alterations in gene expression a result of exposure of a teleost embryo to a consumer product extract. To facilitate observation of alterations in gene expression, a transgenic teleost embryo in which a regulatory sequence of interest (e.g., an inducible promoter) is operably linked to a reporter sequence can be used. The regulatory sequence can be from the fish species under study or a different fish species, as long as it behaves appropriately in the fish species being assayed. Alterations in expression of the reporter following exposure of a consumer product extract as compared to a (negative and/or positive) control can be detected and/or measured.

Suitable reporter sequences will be evident to those of skill in the art. For example, a suitable reporter protein can include fluorescent proteins and enzymes detectable by a histochemical method. The reporter sequences can be introduced into teleost genomes in constructs containing appropriate exogenous regulatory elements (e.g., promoter and 3′ untranslated regions, for example as described in U.S. Pat. No. 9,043,995) or can be knocked into an endogenous genetic locus (for example using the methodology described in Kimura et al., 2014, Scientific Reports 4:6545, doi:10.1038).

Fluorescent proteins are well known in the art. Examples of fluorescent proteins include, without limitation, a green fluorescent protein (GFP), an enhanced green fluorescent protein (EGFP), a red fluorescent protein (CFP and Red FP, RFP), a blue fluorescent protein (BFP), a yellow fluorescent protein (YFP), and fluorescent variants of these proteins. The heterologous fluorescent gene (the term gene in this context refers to any coding sequence, with or without control sequences) may be, for example, a gene encoding DsRed2, ZsGreen1, and ZsYellow1. The heterologous fluorescent gene may encode any naturally occurring or variant marker proteins, including green fluorescent protein (GFP), enhanced green fluorescent protein (eGFP), yellow fluorescent protein (YFP), enhanced yellow fluorescent protein (eYFP), blue fluorescent protein (BFP), enhanced blue fluorescent protein (eBFP), cyan fluorescent protein (CFP), and enhanced cyan fluorescent protein (eCFP).

Enzymes that are detectable by histochemical methods are also well known in the art. Examples of enzymes include, without limitation, luciferase, horseradish peroxidase, β-galactosidase, β-glucuronidase, alkaline phosphatase, chloramphenicol acetyl transferase, and alcohol dehydrogenase. According to a particular embodiment, the enzyme is luciferase. The term “luciferase” is intended to denote all the proteins which catalyze or initiate a bioluminescent reaction in the presence of a substrate called luciferin. The luciferase may be from any organism or system that generates bioluminescence (see, e.g., U.S. Pat. No. 6,152,358). For example, the luciferase may be from Renilla (U.S. Pat. Nos. 5,418,155 and 5,292,658), from Photinus pyralis or from Luciola cruciata (U.S. Pat. No. 4,968,613).

Techniques to detect protein reporters, either directly (e.g., by measuring the amount of reporter mRNA) or indirectly (e.g., by measuring the amount and/or activity of the reporter protein) are conventional. Many of these methodologies and analytical techniques can be found in such references as Current Protocols in Molecular Biology, F. M. Ausubel et al., eds., (a joint venture between Greene Publishing Associates, Inc. and John Wiley & Sons, Inc.), Enzyme Immunoassay, Maggio, ed. (CRC Press, Boca Raton, 1980); Laboratory Techniques in Biochemistry and Molecular Biology, T. S. Work and E. Work, eds. (Elsevier Science Publishers B. V., Amsterdam, 1985); Principles and Practice of Immunoassays, Price and Newman, eds. (Stockton Press, NY, 1991); and the like.

In a particular embodiment, the amount and/or activity of a reporter expression product (e.g., a protein) is measured. A fluorescent marker, such as eGFP, can be detected by detecting its fluorescence in the cell (e.g., in a brackish medaka fish or zebrafish embryo). For example, fluorescence can be observed under a fluorescence microscope and, if desired, can be quantitated. Reporters such as eGFP, which are directly detectable without requiring the addition of exogenous factors, are preferred for detecting or assessing gene expression during fish embryonic development. A transgenic fish embryo engineered to express fluorescent reporter under the control of a promoter of interest can provide a rapid real time in vivo system for analyzing spatial and temporal expression patterns.

4.5.4.1. Endocrine Disruptor Assays

Endocrine disruptors are chemicals that, at certain doses, can interfere with the endocrine (or hormone) system in mammals. These disruptions can cause cancerous tumors, birth defects, and other developmental disorders. Specifically, endocrine disruptors may be associated with the development of learning disabilities, severe attention deficit disorder, cognitive and brain development problems; deformations of the body; breast cancer, prostate cancer, thyroid and other cancers (see Gore et al., 2015, Endocrine Reviews 36(6):593-602. doi: 10.1210/er.2015-1093). One well known example of an endocrine disruptor is bisphenol A, a chemical commonly found in plastic bottles, plastic food containers, dental materials, and the linings of metal food and infant formula cans. Bisphenol A is associated with elevated rates of diabetes, mammary and prostate cancers, decreased sperm count, reproductive problems, early puberty, obesity, and neurological problems.

Endocrine disruptors can be evaluated in transgenic teleost embryos harboring a coding sequence for a marker protein operably linked to a promoter that is sensitive to disruptors of multiple endocrine systems. Because several hormones that operate in different endocrine system share common subunits, the use of a promoter from one of the common subunits permits interrogation of multiple hormone systems simultaneously. One example of such a subunit is the glycoprotein subunit α (gsuα), which encodes the shared a subunit of follicle stimulating hormone β, luteinizing hormone β, and thyroid-stimulating hormone (TSH) β. The gsuα promoter of zebrafish is an example of a promoter that can be operably linked to a coding sequence of a marker protein and used to detect endocrine disrupting chemicals (Cheng et al., 2014, Toxicology and Applied Pharmacology 278:78-84), and can be used to screen for the presence of endocrine disrupting chemicals in consumable products as described herein.

Many endocrine disruptors possess estrogenic, enhancing-estrogenic or anti-estrogenic properties. For the evaluation of the estrogenic, enhancing-estrogenic and anti-estrogenic properties of the consumable product extracts of the disclosure, the consumable product extracts can be assayed in teleost embryos harboring an estrogen responsive promoter operably linked to a coding sequence for a marker protein. In some embodiments, the estrogen responsive promoter is from a choriogenin gene of a medaka fish (e.g., Oryzias melastigma and Oryzias latipes), for example choriogenin H or choriogenin L. Choriogenin H and L are precursor proteins of the inner layer subunits of egg envelope (chorion) of teleost fish, and gene expression of both choriogenin H and choriogenin L are responsive to estrogenic substances (see, e.g., Yamaguchi et al., 2015, J Appl Toxicol. 35(7):752-8). In some embodiments, the choriogenin H promoter is used to assay the estrogen disruptor activity of a consumable product extract. The choriogenin H promoter has been shown to be a highly sensitive biomarker for monitoring estrogenic chemicals in the marine environment (Chen et al., 2008, Ecotoxicol Environ Saf. 71(1):200-8). Examples of choriogenin H promoter constructs suitable for use for assaying estrogenic activity of consumer product extracts are disclosed in U.S. Pat. No. 9,043,995. In other embodiments, the choriogenin L promoter is used. In other embodiments, the estrogen responsive promoter is the brain aromatase B promoter (referred to as a cyp19a1b promoter in zebrafish). The zebrafish cyp19a1b gene exhibits exquisite sensitivity to estrogens and is a sensitive target for estrogen mimics, and has been successfully operably linked to a marker gene such as GFP in transgenic fish (see, e.g., Brion et al., 2012, PLoS ONE 7(5): e36069. doi:10.1371/journal.pone.0036069). In yet another embodiment, the estrogen sensitive promoter is a vitellogenin promoter (for example as described in Schreurs et al., 2004, Environmen. Sci. Technol. 34:4439-44).

Other endocrine disruptors possess androgenic, enhancing-androgenic or anti-androgenic properties. Androgenic, enhancing-androgenic and anti-androgenic properties of consumable product extracts can be evaluated in teleost embryos harboring an androgen responsive promoter operably linked to a coding sequence for a marker protein. In some embodiments, the androgen responsive promoter is the G. aculeatus spiggin promoter, which is responsive to androgens but exhibits no reactivity to, inter alia, estrogens and glucocorticoids (see, e.g., Sebillot et al., 2014, Environ. Sci. Technol. 48:10919-28).

Yet other endocrine disruptors possess thyroid-disrupting properties, e.g., they disrupt the hypothalamic-pituitary-thyroid (HPT) axis. Thyroid/HPT disrupting properties of consumable product extracts can be evaluated in teleost embryos harboring a thyroid hormone (TH) responsive promoter operably linked to a coding sequence for a marker protein. In some embodiments, the thyroid responsive promoter is the thyroid-stimulating hormone subunit β (TSHβ) promoter, which in contrast to other subunits is unique to TSH. Thyroid-stimulating hormone is part of a feedback loop involving TH and thyrotropin-releasing hormone (TRH). Specifically, when low levels of TH are present, TRH is secreted by the hypothalamus to stimulate the release of TSH by the pituitary, which in turn stimulates the thyroid to secrete TH, and the opposite feedback loop occurs when high levels of TH are present. The TSHβ promoter is a useful biomarker for the HPT axis. An example of a TSH β promoter that can be used is the zebrafish TSH β promoter (see, e.g., Ji et al., 2012, Toxicology and Applied Pharmacology 262:149-155.

4.5.4.2. Xenobiotic Assays

Xenobiotics are foreign chemical substances present within an organism. Xenobiotics may be grouped as antioxidants, carcinogens, drugs, environmental pollutants, food additives, hydrocarbons, and pesticides. Pollutants such as dioxins and polychlorinated biphenyls are considered xenobiotics. The body removes xenobiotics by xenobiotic metabolism. This consists of the deactivation and the excretion of xenobiotics, and happens mostly in the liver, by way of reactions catalyzed by the hepatic microsomal cytochrome P450 enzyme system.

For the evaluation of the xenobiotic properties of the consumable product extracts of the disclosure, the consumable product extracts can be assayed in teleost embryos harboring a xenobiotic responsive promoter operably linked to a coding sequence for a marker protein. In some embodiments, the promoter is a cytochrome P450 promoter, e.g., the zebrafish P450 1A (Cyp1a) promoter such as described in Boon and Gong, 2013, PLOS ONE 8(5):e64334.

Xenobiotic properties of food product samples and food product extracts of the disclosure can also be evaluated using an in vivo ethoxyresorufin-O-deethylase (EROD) activity assay using 7-ethoxyresorufin as substrate, for example as described in Liu et al., 2014, Environmental Toxicology 31(2):201-10.

4.5.4.3. Hepatotoxicity Assays

Zebrafish have been studied as models of drug-induced hepatotoxicity. The transparency of zebrafish for several days post-fertilization enables in vivo visual observation of internal organs including liver. Zebrafish complete primary liver morphogenesis by 48 hours post-fertilization (HPF). When exposed to a hepatotoxicant, changes to liver morphology can be evaluated visually (Hill et al., 2012, Drug Metabolism Reviews 44(1):127-140). Researchers have developed various endpoints that can be studied to evaluate hepatotoxicity: liver degeneration, changes in size and shape of the liver, and yolk sac retention (see He et al., 2013, Journal of Pharmacological and Toxicological Methods 67:25-32). These parameters can be assayed in zebrafish to evaluate the hepatotoxicity of a consumable product extract of the disclosure, and analogous parameters can be used to assay the hepatotoxicity of a consumable product extract in a different fish such as medaka.

4.5.4.4. Cardiotoxicity Assays

Teleost embryos provide an ideal model system for investigating cardiotoxicity because their transparency and uncovered hearts make them easily observable. Taking zebrafish as an example, the heart consists of a ventricle and an atrium and these develop rapidly. Heart tube and heartbeat are observed at 24 hours post fertilization (hpf), and then tube looping, chamber formation, and blood circulation are completed by 72 hpf.

It is possible to assay consumable product extracts for cardiotoxicity by evaluating parameters such as heart rate, rhythmicity (e.g., atrioventricular block (AV block), arrhythmia); circulation, and morphology (e.g., pericardial edema; hemorrhage, heart chamber swelling) in teleost embryos.

The heart-specific promoter BMP4 can be used to drive expression of a marker gene that allows heart morphology to be observed. The erythrocyte-specific promoter gata1 can be used to drive expression of a marker gene, allowing the blood circulation rate to be observed (see Wu et al., 2013, Toxicol. Sci. 136(2):402-412, and references cited therein).

These parameters can be assayed in zebrafish or medaka to evaluate the cardiotoxicity of a consumable product extract of the disclosure.

### 5. EXAMPLES

### 5.1. Example 1: Chicken Breast Extract Preparation

Chicken breast produced by farms A, B and C were extracted for acute toxicity and estrogenic activity testing. Chicken breast samples were mechanically homogenized. The homogenized meat were aliquoted and mixed with 1:1.5 (w/v) acetonitrile. After vortexing and sonication, sodium chloride was added until saturation. Samples were centrifuged at 5,000×g for 10 minutes and the supernatant was collected. Anhydrous sodium sulfate was added to the supernatant until saturation. The supernatant was separated and dried under nitrogen gas flow until about 5 ml remained, and then twice washed using 3 ml hexane. The sample was then dried under nitrogen gas flow and redissolved using 200 μl of absolute methanol and stored at −20° C. until testing.

### 5.2. Example 2: Acute Toxicity Testing of Chicken Breast Extract

Extracts prepared as described in Example 1 were tested for acute toxicity using zebrafish (Danio rerio) AB strain embryos. Chicken breast extracts were diluted into zebrafish embryo culture medium at 0.25, 0.50, 1.00, 2.00 and 4.00 μl/ml. Zebrafish AB strain embryos of 4-128 cell stages were exposed to extract dilutions in a 96-well plate at 1 embryo per well. Each concentration was tested with 20 embryos. Zebrafish embryo culture medium and 3.7 mg/L dichloroaniline were included as negative and positive controls, respectively. After 48 hr exposure at 26° C., zebrafish embryos were observed under a stereomicroscope and fish embryos that were coagulated, tail not detached, and having no heart beat were marked as dead. Mortality rate for each concentration was calculated as the acute toxicity endpoint. The mortality rate for the negative control was 0% and for the positive control was 65%. Table 1 shows the acute toxicity test results. Of the three chicken breast extracts, farm A sample extract showed the highest acute toxicity while farm B sample extract showed the lowest toxicity to zebrafish embryos.

TABLE 1
Acute toxicity of chicken breast extracts from farms A, B and C

| | | Tested sample nominal concentration | | | | |
|---|---|---|---|---|---|---|
| | | 19 g/L | 41 g/L | 90 g/L | 197 g/L | 433 g/L |
| Mortality rate | Farm A | 0% | 20% | 100% | 100% | 100% |
| of each sample | Farm B | 5% | 0% | 10% | 10% | 5% |
| | Farm C | 0% | 0% | 35% | 30% | 90% |

### 5.3. Example 3: Estrogenic Activity of Chicken Breast Extract

Extracts prepared as described in Example 1 were tested for estrogenic activity using choriogenin H-eGFP transgenic medaka (Oryzias melastigma) eleutheroembryos generated as described in Example 1 of U.S. Pat. No. 9,043,995. Chicken breast extracts were diluted into medaka (Oryzias melastigma) embryo culture medium (instant ocean salt dissolved in deionized water to make 0.2% salinity) at 2.5 μl/ml. 17β-estradiol was also tested at 1.0, 2.0, 5.0 and 10.0 μg/L as positive controls. Culture medium was tested as negative control. Each concentration contained 3 replicates with each replicate containing 8 eleutheroembryos. After 24-hr exposure at 26° C., eleutheroembryos were observed under green fluorescence microscope and imaged from ventral side using the same imaging setting. Negative control and extracts of samples from farms A and B did not induce observable green fluorescence in the eleutheroembryo livers. The extract of the sample from farm C induced observable green fluorescence in the eleutheroembryo livers.

### 5.4. Example 4: Milk Powder Extract Preparation

Brands A, B and C of formula milk powder for 1-3 year old children were extracted for acute toxicity and estrogenic activity testing. Milk powder was reconstituted with water and mixed with 1:1.5 (v/v) acetonitrile. After vortexing and sonication, sodium chloride was added until saturation and then centrifuged to separate the phases. Anhydrous sodium sulfate was added to the supernatant until saturation. The supernatant was separated and dried under nitrogen gas flow until about 5 ml remained, and then twice washed using 3 ml hexane. The sample was then dried under nitrogen gas flow and re-dissolved using 200 μl of absolute methanol and stored at −20° C. until testing.

### 5.5. Example 5: Acute Toxicity Testing of Milk Powder Extract

The acute toxicity of milk powder extracts prepared as described in Example 4 were tested for acute toxicity using zebrafish (Danio rerio) AB strain embryos. Milk powder extracts were diluted into zebrafish embryo culture medium at 0.33, 0.50, 0.76, 1.74 and 4.00 μl/ml. Zebrafish AB strain embryos of 4-128 cell stages were exposed to extract dilutions in a 96-well plate with 1 embryo per well. Each concentration was tested with 20 embryos. Zebrafish embryo culture medium and 3.7 mg/L 3, 4-dichloroaniline were included as negative and positive controls, respectively. After 48-hr exposure at 26° C., zebrafish embryos were observed under stereomicroscope and fish embryos that were coagulated, tail not detached and having no heart beat were classified as dead. Mortality rate of each concentration was calculated as the acute toxicity endpoint. Mortality rate for negative control was 5% and for positive control was 60%. Table 2 shows the test results. Of the three formula milk extracts, the extract of brand A was the most toxic and the extract of brand C extract was the least toxic to zebrafish embryos.

TABLE 2
Acute toxicity of 3 brands of formula milk extract

| | | Tested sample nominal concentration | | | | |
|---|---|---|---|---|---|---|
| | | 21 g/L | 47 g/L | 103 g/L | 227 g/L | 500 g/L |
| Mortality | Brand A | 5% | 0% | 5% | 100% | 100% |
| rate of | Brand B | 0% | 10% | 0% | 0% | 100% |
| each sample | Brand C | 0% | 5% | 0% | 15% | 15% |

### 5.6. Example 6: Estrogenic Activity of Milk Powder Extract

The estrogenic activity of milk powder extracts prepared as described in Example 4 were tested using choriogenin H-EGFP transgenic medaka (Oryzias melastigma) eleutheroembryos. Milk powder extracts were diluted into medaka (Oryzias melastigms) embryo culture medium (instant ocean salt dissolved in deionized water to make 0.2% salinity) at 2.5 μl/ml. 17β-estradiol was also tested at 1.0, 2.0, 5.0 and 10.0 μg/L as positive controls. Culture medium was tested as negative control. Each concentration contained 3 replicates with each replicate contained 8 eleutheroembryos. After 24-hr exposure at 26° C., eleutheroembryos were observed under green fluorescence microscope and imaged from ventral side using the same imaging setting. Extracts of samples from brand A and brand C did not induce observable green fluorescence in the eleutheroembryo livers, while the extract of formula milk of brand B did induce observable green fluorescence in the eleutheroembryo livers. Table 3 shows milk powder estrogenic activity data. The estrogen equivalent concentration in Table 3 means the estrogen activity of a sample equivalent to that of 17 beta-estradiol.

TABLE 3
Estrogen equivalent concentration of 3 baby milk formula extract

| | | |
|---|---|---|
| Estrogen equivalent concentration | Brand A | Not detected |
| of each sample | Brand B | 20 ng/g |
| | Brand C | 30 ng/g |

### 5.7. Example 7: Assay for Cooking Oil

1 volume of cooking oil sample (lard or peanut oil) was mixed with 1:1.5 (v/v) of each of (1) acetone, (2) hexane (3) acetonitrile, (4) methanol, and (5) ethanol. After the resulting mixture was vortexed and centrifuged, the supernatant layers of mixtures (3)-(5) were collected and then dried under nitrogen gas flow. It was found that the solvents (1)-(2) in the mixtures cannot be separated from oil, so they both cannot be used in the assay. However, mixture (5) contains the most oil, whereas mixture (3) contains the least oil, so mixture (3) was selected to be used in the assay. Other solvents like toluene, ether, dichloromethane and chloroform were also used and are applicable to this step.

The solvent in mixture (3), acetonitrile, is water miscible and sodium chloride can help to separate water from the mixture. Magnesium sulfate, calcium chloride, magnesium chloride, sodium acetate, ammonium acetate and sucrose can also be used. After vortexing and sonication, sodium chloride was added to the supernatant (3) until saturation so that the water contained therein could be separated. The resulting sample was further subjected to vortexing and sonication and the supernatant was collected. Anhydrous sodium sulfate (or magnesium sulfate, sodium sulfate, calcium chloride or calcium sulfate) was added to the supernatant until saturation. The supernatant was separated and dried under nitrogen gas flow until about 5 ml remained, and then twice washed using 3 ml hexane. The resulting supernatant was then dried under nitrogen gas flow and redissolved using 200 μl of absolute methanol and stored at −20° C. until testing.

The estrogenic activity and acute toxicity testing of the cooking oil samples were conducted according to the methods described in the above sections 5.5 (Example 5) and 5.6 (Example 6) and the results show that it can be identified whether a toxicant is present in the cooking oil. The acute toxicity data of lard extract and peanut oil extract are shown in Table 4 and Table 5 below.

TABLE 4
Acute toxicity of 3 lard extract

| | | Tested sample nominal concentration | | | | |
|---|---|---|---|---|---|---|
| | | | | 90 | 197 | 433 |
| | | 19 mL/L | 41 mL/L | mL/L | mL/L | mL/L |
| Mortality rate | Brand A | 0% | 0% | 5% | 0% | 205 |
| of | Brand B | 5% | 40% | 100% | 100% | 100% |
| each sample | Brand C | 100% | 100% | 100% | 100% | 100% |

Brand A: known well refined lard from good origin
Brand B: known tainted (gutter)
Brand C: known unrefined lard

TABLE 5
Acute toxicity of 3 peanut oil extract

| | | Tested sample nominal concentration | | | | |
|---|---|---|---|---|---|---|
| | | | | 90 | 197 | 433 |
| | | 19 mL/L | 41 mL/L | mL/L | mL/L | mL/L |
| Mortality rate | Brand A | 0% | 15% | 20% | 35% | 0% |
| of | Brand B | 35% | 80% | 100% | 100% | 35% |
| each sample | Brand C | 100% | 100% | 100% | 100% | 100% |

### 5.8. Example 8: Assay for Drink

Three soy milk sample of different brands (A, B and C) were extracted for toxicity testing. Acetonitrile was added to the sample at a ratio of 1.5:1 (v/v) to obtain an acetonitrile extract. The extract was dried under nitrogen gas flow and redissolved using methanol and stored at −20° C. until testing.

The estrogenic activity and acute toxicity testing of three drink sample extract were conducted according to the methods described in the above sections 5.5 (Example 5) and 5.6 (Example 6) and the results show that it can be identified whether a toxicant is present in the soy milk. The acute toxicity data and the estrogen equivalent concentration of the soy milk extracts are shown in Table 6 and Table 7 below.

TABLE 6
Acute toxicity of 3 soy milk extract

| | | Tested sample nominal concentration | | | | |
|---|---|---|---|---|---|---|
| | | 21 g/L | 47 g/L | 103 g/L | 227 g/L | 500 g/L |
| Mortality | Brand A | 0% | 0% | 5% | 0% | 10% |
| rate of | Brand B | 0% | 2% | 60% | 100% | 100% |
| each sample | Brand C | 0% | 10% | 20% | 60% | 85% |

TABLE 7
Estrogen equivalent concentration of 3 soy milk extract

| | | |
|---|---|---|
| Estrogen equivalent | Brand A | Not detected |
| concentration of each | Brand B | Not detected |
| sample | Brand C | 30 ng/mL |

### 5.9. Example 9: Lipid-Containing Water-Saturated Solid/Semi-Solid Samples (e.g. Yogurt)

The yogurt samples were homogenized. Acetonitrile was added to the sample in a ratio of 1.5:1 (v/w) to obtain an acetonitrile extract. Anhydrous Na₂SO₄ was added the acetonitrile extracts to remove water. The resulting extract was dried under nitrogen gas flow and re-dissolved using methanol and stored at −20° C. until testing.

The estrogenic activity and acute toxicity testing of three yogurt sample extract were conducted according to the methods described in the above sections 5.5 (Example 5) and 5.6 (Example 6) and the results show that it can be identified whether a toxicant is present in the yogurt. The acute toxicity data and the estrogen equivalent concentration of the yogurt extracts are shown in Table 8 and Table 9 below.

TABLE 8
Acute toxicity of 3 yogurt extract

| | | Tested sample nominal concentration | | | | |
|---|---|---|---|---|---|---|
| | | 21 g/L | 47 g/L | 103 g/L | 227 g/L | 500 g/L |
| Mortality | Brand A | 0% | 25% | 59% | 100% | 10% |
| rate of | Brand B | 0% | 10% | 30% | 60% | 100% |
| each sample | Brand C | 0% | 10% | 0% | 30% | 65% |

TABLE 9
Estrogen equivalent concentration of 3 yogurt extract

| | | |
|---|---|---|
| Estrogen equivalent | Brand A | Not detected |
| concentration of each | Brand B | Not detected |
| sample | Brand C | 14 ng/g |

### 5.10. Example 10: Lipid-Containing Water-Unsaturated Solid/Semi-Solid Samples (e.g. Wheat Powder)

The wheat powder samples were homogenized. Water was added to the resulting sample to form a mixture. Acetonitrile was added to the mixture at a ratio of 1.5:1 (v/v) to obtain an acetonitrile extract. NaCl was added the acetonitrile extract to saturate the water. Anhydrous Na₂SO₄ was added to the resulting extract to remove water and then hexane was added to the extract to remove lipid. The resulting extract was dried under nitrogen gas flow and redissolved using methanol and stored at −20° C. until testing.

The estrogenic activity and acute toxicity testing of three wheat powder sample extract were conducted according to the methods described in the above sections 5.5 (Example 5) and 5.6 (Example 6) and the results show that it can be identified whether a toxicant is present in the wheat powder. The acute toxicity data and the estrogen equivalent concentration of the wheat powder extracts are shown in Table 10 and Table 11 below.

TABLE 10
Acute toxicity of 3 wheat powder extract

| | | Tested sample nominal concentration | | | | |
|---|---|---|---|---|---|---|
| | | 21 g/L | 47 g/L | 103 g/L | 227 g/L | 500 g/L |
| Mortality | Brand A | 0% | 5% | 0% | 0% | 10% |
| rate of | Brand B | 0% | 0% | 10% | 20% | 30% |
| each sample | Brand C | 0% | 5% | 15% | 30% | 65% |

TABLE 11
Estrogen equivalent concentration of 3 wheat powder extract

| | | |
|---|---|---|
| Estrogen equivalent | Brand A | Not detected |
| concentration of each | Brand B | Not detected |
| sample | Brand C | Not detected |

### 5.11. Non-Lipid-Containing Water-Saturated Solid/Semi-Solid Samples (e.g. Jam)

The jam samples were homogenized. Acetonitrile was added to the sample at a ratio of 1:1 (v/w) to obtain an acetonitrile extract. NaCl was added to the acetonitrile extract to saturate the water. Anhydrous Na2SO4 was added to the resulting extract to remove water. The resulting extract was dried under nitrogen gas flow and re-dissolved using methanol and stored at −20° C. until testing.

The estrogenic activity and acute toxicity testing of three jam sample extract were conducted according to the methods described in the above sections 5.5 (Example 5) and 5.6 (Example 6) and the results show that it can be identified whether a toxicant is present in the jam. The acute toxicity data and the estrogen equivalent concentration of the jam extracts are shown in Table 12 and Table 13 below.

TABLE 12
Acute toxicity of 3 jam extract

| | | Tested sample nominal concentration | | | | |
|---|---|---|---|---|---|---|
| | | 21 g/L | 47 g/L | 103 g/L | 227 g/L | 500 g/L |
| Mortality | Brand A | 0% | 5% | 0% | 70% | 100% |
| rate of | Brand B | 0% | 0% | 0% | 20% | 70% |
| each sample | Brand C | 0% | 50% | 100% | 100% | 100% |

TABLE 13
Estrogen equivalent concentration of 3 jam extract

| | | |
|---|---|---|
| Estrogen equivalent | Brand A | Not detected |
| concentration of each | Brand B | Not detected |
| sample | Brand C | Not detected |

### 5.12. Non-Lipid-Containing Water-Unsaturated Solid/Semi-Solid Samples (e.g. Animal Feed)

The feed sample was homogenized. Acetonitrile was added to the sample at a ratio of 1.5:1 (v/w) to obtain an acetonitrile extract. NaCl was added to the acetonitrile extract to saturate the water. The resulting extract was added with anhydrous Na₂SO₄ to remove water. The resulting extract was dried under nitrogen gas flow and redissolved using methanol and stored at −20° C. until testing.

The estrogenic activity and acute toxicity testing of three duck feed sample extract were conducted according to the methods described in the above sections 5.5 (Example 5) and 5.6 (Example 6) and the results show that it can be identified whether a toxicant is present in the duck feed. The acute toxicity data and the estrogen equivalent concentration of the duck feed extracts are shown in Table 14 and Table 15 below.

TABLE 14
Acute toxicity of 3 duck feed extract

| | | Tested sample nominal concentration | | | | |
|---|---|---|---|---|---|---|
| | | 21 g/L | 47 g/L | 103 g/L | 227 g/L | 500 g/L |
| Mortality | Brand A | 0% | 5% | 10% | 70% | 90% |
| rate of | Brand B | 0% | 0% | 30% | 70% | 100% |
| each sample | Brand C | 10% | 60% | 80% | 100% | 100% |

TABLE 15
Estrogen equivalent concentration of 3 duck feed extract

| | | |
|---|---|---|
| Estrogen equivalent | Brand A | Not detected |
| concentration of each | Brand B | 21 ng/g |
| sample | Brand C | 91 ng/g |

### 5.13. Lipid-Containing Liquid Samples (e.g. Liquid Milk)

Acetonitrile was added to a liquid milk sample at a ratio of 1.5:1 (v/v) to obtain an acetonitrile extract. NaCl was added to the acetonitrile extract to saturate the water. Anhydrous Na₂SO₄ was added to the resulting extract to remove water. The resulting extract was dried under nitrogen gas flow and redissolved using methanol and stored at −20° C. until testing.

The estrogenic activity and acute toxicity testing of three liquid milk sample extract were conducted according to the methods described in the above sections 5.5 (Example 5) and 5.6 (Example 6) and the results show that it can be identified whether a toxicant is present in the liquid milk. The acute toxicity data and the estrogen equivalent concentration of the liquid milk extracts are shown in Table 16 and Table 17 below.

TABLE 16
Acute toxicity of 3 liquid milk extract

| | | Tested sample nominal concentration | | | | |
|---|---|---|---|---|---|---|
| | | | | 207 | 455 | 1000 |
| | | 43 mL/L | 94 mL/L | mL/L | mL/L | mL/L |
| Mortality rate | Brand A | 0% | 5% | 5% | 0% | 30% |
| of | Brand B | 0% | 0% | 20% | 60% | 100% |
| each sample | Brand C | 100% | 100% | 100% | 100% | 100% |

TABLE 17
Estrogen equivalent concentration of 3 liquid milk extract

| | | |
|---|---|---|
| Estrogen equivalent | Brand A | 3 ng/g |
| concentration of each | Brand B | Not detected |
| sample | Brand C | Not detected |

### 5.14. Non-Lipid-Containing Liquid Samples (e.g. Juice)

Acetonitrile was added to liquid milk sample at a ratio of 1.5:1 (v/v) to obtain an acetonitrile extract. NaCl was added to the acetonitrile extract to saturate the water. Anhydrous Na₂SO₄ was added to ₜhe resulting extract to remove water. The resulting extract was dried under nitrogen gas flow and re-dissolved using methanol and stored at −20° C. until testing.

The estrogenic activity and acute toxicity testing of three grape juice sample extract were conducted according to the methods described in the above sections 5.5 (Example 5) and 5.6 (Example 6) and the results show that it can be identified whether a toxicant is present in the grape juice. The acute toxicity data and the estrogen equivalent concentration of the grape juice extracts are shown in Table 18 and Table 19 below.

TABLE 18
Acute toxicity of 3 grape juice extract

| | | Tested sample nominal concentration | | | | |
|---|---|---|---|---|---|---|
| | | | | 207 | 455 | 1000 |
| | | 43 mL/L | 94 mL/L | mL/L | mL/L | mL/L |
| Mortality rate | Brand A | 0% | 5% | 5% | 0% | 0% |
| of | Brand B | 0% | 0% | 10% | 0% | 10% |
| each sample | Brand C | 0% | 0% | 20% | 40% | 60% |

TABLE 19
Estrogen equivalent concentration of 3 grape juice extract

| | | |
|---|---|---|
| Estrogen equivalent | Brand A | Not detected |
| concentration of each | Brand B | Not detected |
| sample | Brand C | Not detected |

### 5.15. Lard Sample

The acute toxicity (mortality at 48 hours of exposure) of extracts of 7 lard samples of known quality (state of refinement and purity) were tested with zebrafish embryos. Cut-off criteria for acceptance of toxicity data were set when the mortality rates of zebrafish embryos in the blank (culture medium) and solvent (0.4% methanol) controls were ≦10% and >30% in the positive control (3.7 mg/L 3, 4-dichloroaniline). The LC₅₀ of the extract of each oil was calculated based on the embryo mortality rate versus dose response curve, and presented as the nominal concentration of the original oil. Table 20 shows that normally-produced lards (LN1 and LN2) exhibited low toxicity (LC₅₀>173.3 mL/L), whilst unrefined lard (LT1 and LT2) exhibited high toxicity (LC₅₀<14.3 mL/L) and the remaining lards, which were not well-refined, exhibited varying toxicity, with LC₅₀s between 14.3 mL/L and 173.3 mL/L.

TABLE 20
Individual lard sample descriptors and respective acute toxicity values in
zebrafish embryos

| Sample Code^{a} | Description | LC50 (mL/L) |
|---|---|---|
| LT1 | Tainted oil-Raw lard (unrefined) | <14.3 |
| LT2 | Tainted oil-Raw lard (unrefined) | <14.3 |
| LT3 | Tainted oil-Refined lard | 21.7 |
| LT4 | Tainted oil-Refined lard | 38.6 |
| LT5 | Tainted oil-Not proper refined oil | 48.3 |
| LN1 | Normal oil-Raw lard | >173.3 |
| LN2 | Normal lard-Raw lard | >173.3 |

^{a}LT, Tainted lard; LN, Normal lard.

To determine potential correlations between the zebrafish embryo toxicity test results and potential chemical contaminants, acetonitrile extracts of representative lards of low (LN1, LC50>173.3 mL/L), moderate (LT5, LC50=48.3 mL/L) and high (LT1, LC50<14 3 mL/L) toxicity, as classified by the bioassay, were subjected to non-targeted, high resolution LCMS analysis, with accompanying multivariate statistical analysis. The PCA analysis of m/z signals obtained for each lard extract tested, with clear separation based on the acute toxicity of the extract. Further to this, a total of 7 (positive ion mode) and 9 (negative ion mode) characteristic m/z signals (intensities between 293.2109 and 445.2786) were selected from the spectra of the high toxicity lard (LT1) for further identification (Table 21). In contrast to this, the signal intensities of LT5 and LN1 were approximately 10-100% and 0-20% that of LT1, respectively. Indeed, the low acute toxicity sample (LN1) was similar to the blank, indicating that the selected m/z features correlated positively with the zebrafish embryo toxicity test results. Furthermore, the identification results from the Progenesis QI software confirmed that most of the signals (i.e., all except m/z 354.2850 and 353.2299) matched the corresponding compounds (Table 21), identified by ChemSpider. In the negative ion mode, the possible compound candidates of m/z 311.2214, 309.52507 and 329.2319 were all lipid oxidation products (Table 22).

TABLE 21
Chemical descriptor characteristics of selected m/z ions obtained from
acetonitrile extracts of the most toxic lard (LT1) and their relative signal intensities in other
tested lard samples of lower toxicity^{a}.

| | | | | Signal | | | |
|---|---|---|---|---|---|---|---|
| Information of characteristic signals | | | | intensity | Relative signal intensity (%) | | |
| | major | neutral | chemical | high toxicity | moderate | low toxicity | |
| m/z | adduct | mass (Da) | formula | LT1 | toxicity LT5 | LN1 | blank |
| Positive | | | | | | | |
| 408.2929 | M + Na | 385.3037 | C₁₉H₃₉N₅O₃ | 7.70E+05 | 25.7 ± 0.6 | 0.6 ± 0.1 | 0.4 ± 0.1 |
| 406.2747 | M + Na | 383.2853 | C₂₆H₄₀P | 6.29E+05 | 26.1 ± 2.1 | 0.9 ± 0.0 | 0.8 ± 0.1 |
| 341.2054 | M + Na | 318.2162 | C₁₆H₂₆N₆O | 5.07E+05 | 18.4 ± 0.9 | 5.5 ± 0.5 | 4.1 ± 0.2 |
| 393.2611 | M + Na | 370.2719 | C₂₁H₃₈O₅ | 3.73E+05 | 19.2 ± 0.4 | 6.2 ± 0.7 | 1.6 ± 0.2 |
| 357.2006 | M + Na | 334.2114 | C₁₆H₂₆N₆O₂ | 3.32E+05 | 41.5 ± 1.4 | 1.2 ± 0.5 | 0.3 ± 0.3 |
| 354.2850 | M + NH₄ | 336.2513 | No match | 4.25E+05 | 18.2 ± 0.6 | 5.8 ± 2.4 | 1.6 ± 0.0 |
| 353.2299 | M + Na | 330.2408 | No match | 3.67E+05 | 14.8 ± 1.4 | 3.4 ± 0.9 | 4.4 ± 0.9 |
| Negative | | | | | | | |
| 311.2214 | M − H | 312.2287 | C₁₈H₃₂O₄ | 1.75E+06 | 101.1 ± 7.0 | 5.1 ± 0.1 | 4.1 ± 0.5 |
| 299.2578 | M − H | 300.2650 | C₁₈H₃₆O₃ | 1.22E+06 | 73.6 ± 2.1 | 16.6 ± 3.3 | 1.5 ± 0.3 |
| 327.2162 | M − H | 328.2235 | C₁₆H₃₀N₃O₄ | 1.14E+06 | 46.7 ± 5.1 | 5.0 ± 0.2 | 3.7 ± 0.1 |
| 393.2240 | M + Na − 2H | 372.2493 | C₂₀H₃₆O₆ | 9.68E+05 | 10.4 ± 0.7 | 1.0 ± 0.1 | 0.9 ± 0.1 |
| 309.2057 | M − H | 310.2137 | C₁₈H₃₀O₄ | 9.03E+05 | 84.4 ± 2.6 | 4.6 ± 0.4 | 3.5 ± 0.4 |
| 329.2319 | M − H | 330.2399 | C₁₈H₃₄O₅ | 8.50E+05 | 37.2 ± 2.5 | 5.7 ± 0.3 | 6.1 ± 0.5 |
| 393.2243 | M + FA − H | 348.2260 | C₁₆H₃₂N₂O₆ | 6.32E+05 | 94.8 ± 5.1 | 8.2 ± 0.1 | 5.9 ± 0.7 |
| 293.2109 | M − H | 294.2181 | C₁₈H₃₀O₃ | 5.49E+05 | 71.2 ± 1.8 | 3.8 ± 0.6 | 1.2 ± 0.2 |
| 445.2786 | M + Na − 2H | 424.3016 | C₁₈H₃₆N₁₀O₂ | 4.73E+05 | 29.0 ± 0.5 | 11.5 ± 0.9 | 0.3 ± 0.2 |

^{a}The m/z order is dependent on the signal intensity of LT1.

TABLE 22
Suggested structural identities of some m/z ions with strong
correlation to the induction of acute toxicity of lard extracts in zebrafish embryos.

| | Major | Neutral mass | | Probable chemical compound | |
|---|---|---|---|---|---|
| m/z | adduct | (Da) | Formula | Name | Structure |

Negative ion mode

| | | | | | |
|---|---|---|---|---|---|
| 311.2214 | M—H | 312.2287 | C₁₈H₃₂O₄ | 8-{3-[(1E)-3-Hydroxy- 1-octen-1-yl]-2-oxiranyl} octanoic acid | 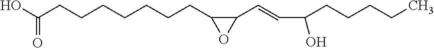 [SMILES: CCCCCC(O)/C=C/C1OC1CCCCCCCC(=O)O] |
| 309.2057 | M—H | 310.2144 | C₁₈H₃₀O₄ | 9-Hydroxy-11-(3-pentyl- 2-oxiranyl)-7,10- undecadienoic acid | 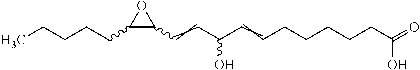 [SMILES: CCCCCC1OC1C=CC(O)C=CCCCCCC(=O)O] |
| 329.2319 | M—H | 330.2406 | C₁₈H₃₄O₅ | 9,10,13-Trihydroxy-11- 1-octadecenoic acid | 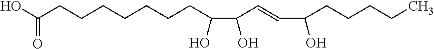 [SMILES: CCCCCC(O)/C=C/C(O)C(O)CCCCCCCC(=O)O] |
| | | | | | 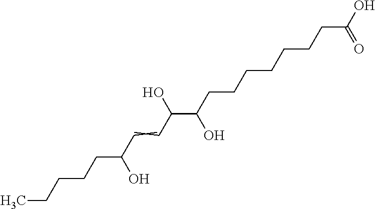 [SMILES: CCCCCC(O)C=CC(O)C(O)CCCCCCCC(=O)O] |
| | | | | | 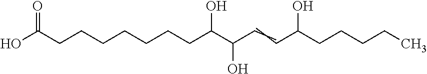 [SMILES: CCCCCC(O)C=CC(O)C(O)CCCCCCCC(=O)O] |
| | | | | Ethyl (6R)-6-hydroxy-6-{(2R,5R)- 5-[(1S)-1-hydroxyhexyl] tetrahydro-2-furanyl} hexanoate | 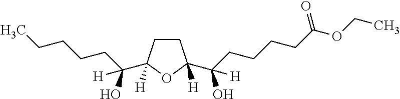 [SMILES: CCCCC[C@H](O)[C@H]1CC[C@H]([C@H](O)CCCCC(=O)OCC)O1] |

### 5.16. Peanut Oil Sample

The acute toxicity of 6 peanut oils from Mainland China and 3 peanut oils from Hong Kong were tested in this study. Table 23 shows that the acetonitrile extracts of these 9 peanut oils exerted varying acute toxicity in zebrafish embryo cultures, with LC₅₀s ranging from <14.3 mL/L to >173.3 mL/L The extract of sample P1 showed the highest acute toxicity with an LC₅₀<14 3 mL/L, whereas extracts of samples P7, P8 and P9 showed the lowest acute toxicity with LC₅₀s>173.3 mL/L. Extracts of the other 5 samples showed varying toxicity, with LC₅₀s between 14.3 mL/L and 173.3 mL/L. No obvious correlation between the description of state of purity of the oil and the respective LC₅₀ values was identified.

TABLE 23
Individual peanut oil sample descriptors and respective acute toxicity
values in zebrafish embryos

| Sample | | | |
|---|---|---|---|
| Code | Description | Source | LC50 (ml/L) |
| P1 | Peanut oil | China market | <14.3 |
| P2 | Traditional method | China market | 20.0 |
| | squeezed peanut oil | | |
| P3 | Peanut oil | China market | 95.3 |
| P4 | Peanut oil | Hong Kong market | 104.2 |
| P5 | Five techniques processed | China market | 133.0 |
| | peanut oil | | |
| P6 | Peanut oil | China market | 140.0 |
| P7 | Refined peanut oil | China market | >173.3 |
| P8 | Pure peanut oil | Hong Kong market | >173.3 |
| P9 | Peanut oil | Hong Kong market | >173.3 |

The same procedure to that used with lard was used to determine if there was any correlation between the toxicity potential and chemical composition of peanut oils. The PCA scatter plot shows that oil extracts with varying toxicity can be clustered and separated based on the m/z signals from high resolution LC/MS analysis. However, there were no correlations in the characteristic chemical signals obtained from the most toxic peanut oil (P1, Table 24) and those obtained from the most toxic lard sample (LT1, Table 21). A total of 4 (positive ion mode) and 2 (negative ion mode) characteristic m/z signals were selected (Table 24), ranging between 165.0912 and 628.1956. The signal intensities of the low (P7) and moderately (P2) toxic peanut oils were approximately 10-90% and 0-20% that of P1 respectively, indeed m/z 299.1102, 195.1017 and 165.0912 all exhibited intensities <5% in the low and moderately toxic samples. The identification results from the Progenesis QI software, confirmed that most of the signals (except m/z 628.1956) matched the corresponding compounds listed in ChemSpider. However, unlike the lards, no lipid oxidation products were identified.

TABLE 24
Chemical descriptor characteristics of selected m/z ions obtained from acetonitrile
extracts of the most toxic peanut oil (P1) and their relative signal intensities in other tested peanut
oil samples of lower toxicity^{a}.

| | | | | Signal | | | |
|---|---|---|---|---|---|---|---|
| Information of characteristic signals | | | | intensity | Relative signal intensity (%) | | |
| | major | neutral | chemical | high toxicity | moderate toxicity | low toxicity | |
| m/z | adduct | mass (Da) | formula | P1 | P2 | P7 | blank |
| Positive | | | | | | | |
| 397.1986 | M + Na | 374.2094 | C₁₈H₂₅FN₇O | 3.88E+05 | 52.3 ± 5.1 | 1.3 ± 0.0 | 0.0 ± 0.0 |
| 299.1102 | M + Na | 276.1212 | C₁₁H₁₅F₃N₄O | 3.85E+05 | 3.3 ± 0.3 | 4.7 ± 0.3 | 0.7 ± 0.1 |
| 195.1017 | M + CH₃OH + H | 162.0681 | C₁₀H₁₀O₂ | 2.50E+05 | 1.8 ± 0.5 | 1.7 ± 0.4 | 2.0 ± 1.0 |
| 628.1956 | M + NH₄ | 610.1617 | No match | 6.89E+06 | 87.9 ± 11.6 | 17.1 ± 1.0 | 2.8 ± 1.0 |
| Negative | | | | | | | |
| 317.1379 | M − H | 318.1452 | C₁₈H₂₂O₅ | 2.76E+05 | 8.2 ± 0.3 | 1.7 ± 0.2 | 0.0 ± 0.0 |
| 165.0912 | M − H | 166.0985 | C₆H₁₇NO₂P | 2.41E+05 | 0.0 ± 0.0 | 0.0 ± 0.0 | 0.2 ± 0.1 |

^{a}The m/z order is dependent on the signal intensity of P1.

### 6. SPECIFIC EMBODIMENTS

The present disclosure is exemplified by the specific embodiments below.

1. A method of determining whether a toxicant is present in a consumable product, comprising:
a) combining a polar organic solvent and the consumable product to obtain a polar organic extract with an overall toxicity;
b) contacting a teleost embryo with the organic extract of a);
c) determining whether the extract exerts a toxicity effect on the embryo;
wherein a toxicity effect on the embryo shows an overall toxicity of the consumable product.

2. The method of embodiment 1, wherein the consumable product comprises a food or a beverage.

3. The method of embodiment 2, wherein the consumable product comprises a food, optionally an edible oil, a human food, a pet food, or a livestock feed.

4. The method of embodiment 3, in which the food is selected from an edible oil, a packaged food, a dairy product, meat, wheat powder, yogurt, lard, peanut oil, and infant formula.

5. The method of embodiment 4, in which the food is a dairy product selected from milk, cream, yogurt, ice cream, jam, butter, and cheese.

6. The method of embodiment 4, in which the food is a meat selected from beef, chicken, pork, fish, duck, and lamb.

7. The method of any one of embodiments 1 to 6, wherein the extract is an organic solvent extract.

8. The method of embodiment 7, wherein the organic solvent comprises acetonitrile, methanol, ethanol, acetone, toluene, diethyl ether, dichloromethane, chloroform, hexane or a mixture thereof.

9. The method of embodiment 8, in which the consumable product comprises a food and the organic solvent comprises acetonitrile.

10. The method of any one of embodiments 7 to 9, wherein the organic solvent extract is dehydrated.

11. The method of embodiment 10, wherein the organic solvent extract is dehydrated by one or more steps to remove water.

12. The method of any one of embodiments 1 to 11, wherein the extract is delipidated when the sample contains lipid or lipid rich. For non or low lipid containing samples, no delipidation is needed.

13. The method of embodiment 12, wherein the extract is obtainable by a process comprising the step of a hexane wash.

14. The method of embodiment 7 or embodiment 8, wherein the organic solvent extract is obtainable by a process comprising:
a) combining the sample with the organic solvent and, optionally, a first salt and/or a sugar to form a mixture; and
b) separating a phase containing the organic solvent from the mixture, thereby obtaining an organic solvent extract.

15. The method of embodiment 14, in which step (a) comprises combining the sample with the organic solvent and a first salt and/or a sugar.

16. The method of embodiment 15, in which step (a) comprises combining the sample with the organic solvent and a first salt.

17. The method of embodiment 16, wherein the first salt comprises sodium chloride, magnesium sulfate, sodium sulfate, calcium sulfate, calcium chloride, magnesium chloride, sodium acetate, ammonium acetate, anhydrous sodium sulfate, anhydrous magnesium sulfate, anhydrous calcium chloride, anhydrous calcium sulfate, or a combination thereof.

18. The method of embodiment 17, wherein the first salt comprises a combination of (i) sodium chloride, magnesium sulfate, calcium chloride, magnesium chloride, sodium acetate, ammonium acetate, or a combination thereof, and (ii) anhydrous sodium sulfate, anhydrous magnesium sulfate, anhydrous sodium sulfate, anhydrous calcium sulfate, anhydrous calcium chloride, anhydrous calcium sulfate, or a combination thereof.

19. The method of embodiment 16, wherein the first salt comprises sodium chloride, magnesium sulfate, calcium chloride, magnesium chloride, sodium acetate, ammonium acetate, or a combination thereof.

20. The method of embodiment 15, in which step (a) comprises combining the sample with the organic solvent and a sugar, optionally wherein the sugar comprises sucrose.

21. The method of embodiment 14, in which step (a) comprises combining the sample with the organic solvent but not with a first salt or a sugar.

22. The method of embodiment 21, wherein the process further comprises a step of combining the mixture with a first salt and/or a sugar prior to step (b).

23. The method of embodiment 22, wherein the process further comprises a step of mixing the mixture before combining the mixture with a first salt and/or a sugar.

24. The method of embodiment 23, wherein mixing the mixture comprises vortexing the mixture, sonicating the mixture, or a combination thereof.

25. The method of any one of embodiments 22 to 24, wherein the process comprises combining the mixture with a first salt prior to step (b).

26. The method of embodiment 25, wherein the first salt comprises sodium chloride, magnesium sulfate, calcium chloride, magnesium chloride, sodium acetate, ammonium acetate, anhydrous sodium sulfate, anhydrous magnesium sulfate, anhydrous calcium chloride, anhydrous calcium sulfate, or a combination thereof.

27. The method of embodiment 26, wherein the first salt comprises a combination of (i) sodium chloride, magnesium sulfate, calcium chloride, magnesium chloride, sodium acetate, ammonium acetate, or a combination thereof, and (ii) anhydrous sodium sulfate, anhydrous magnesium sulfate, anhydrous calcium chloride, anhydrous calcium sulfate, or a combination thereof.

28. The method of embodiment 25, wherein the first salt comprises sodium chloride, magnesium sulfate, calcium chloride, magnesium chloride, sodium acetate, ammonium acetate, or a combination thereof.

29. The method of any one of embodiments 22 to 24, wherein the process comprises combining the mixture with a sugar prior to step (b), optionally wherein the sugar comprises sucrose.

30. The method of any one of embodiments 14 to 29, wherein the process further comprises a step of mixing the mixture prior to step (b).

31. The method of embodiment 30, wherein mixing the mixture prior to step (b) comprises vortexing the mixture, sonicating the mixture, or a combination thereof.

32. The method of any one of embodiments 14 to 31, wherein step (b) comprises centrifuging the mixture to separate the phase containing the organic solvent from the mixture.

33. The method of any one of embodiments 14 to 32, wherein the process further comprises:
c) adding a second salt to the phase containing the organic solvent from step (b) to form a second mixture; and
d) separating a phase containing the organic solvent from the second mixture.

34. The method of embodiment 33, wherein the second salt comprises anhydrous sodium sulfate, anhydrous magnesium sulfate, anhydrous calcium sulfate, anhydrous calcium chloride, or a combination thereof.

35. The method of embodiment 33 or embodiment 34, wherein the process further comprises a step of mixing the second mixture before step (d).

36. The method of embodiment 35, wherein mixing the second mixture comprises vortexing the second mixture, sonicating the second mixture, or a combination thereof.

37. The method of any one of embodiments 33 to 36, wherein step (d) comprises centrifuging the second mixture to separate the phase containing the organic solvent from the second mixture.

38. The method of any one of embodiments 14 to 37, wherein the process further comprises a step of homogenizing the sample prior to step (a).

39. The method of any one of embodiments 14 to 38, wherein the organic solvent is other than hexane and the process further comprises washing the organic solvent extract at least once with hexane.

40. The method of any one of embodiments 14 to 39, wherein the organic solvent comprises acetonitrile.

41. The method of any one of embodiments 14 to 40, wherein the process further comprises removing the organic solvent from the organic solvent extract and redissolving the organic solvent extract in a second organic solvent.

42. The method of embodiment 41, wherein the second organic solvent comprises methanol, dimethyl sulfoxide, or a combination thereof.

43. The method of any one of embodiments 14 to 42, wherein the process further comprises recovering the organic solvent extract.

44. The method of any one of embodiments 1 to 43, which further comprises preparing the extract.

45. The method of embodiment 44, wherein the extract is prepared by a process comprising the steps described in any one of embodiments 14 to 43.

46. The method of embodiment 45, in which the extract is an acetonitrile extract and wherein the extract is prepared by a process in which step (a) of the process comprises combining the sample with acetonitrile.

47. The method of embodiment 46, wherein the extract is prepared by a process in which step (a) of the process comprises combining the sample with acetonitrile and sodium chloride.

48. The method of embodiment 46, wherein the extract is prepared by a process in which the mixture is combined with sodium chloride prior to step (b) of the process.

49. The method of any one of embodiments 46 to 48, wherein the extract is prepared by a process in which the mixture is mixed before step (b).

50. The method of any one of embodiments 46 to 49, wherein the extract is prepared by a process comprising adding anhydrous sodium sulfate to the phase containing the acetonitrile from step (b) to form a second mixture.

51. The method of embodiment 50, wherein the extract is prepared by a process comprising a step of mixing the second mixture and separating a phase containing the acetonitrile from the second mixture.

52. The method of any one of embodiments 46 to 51, wherein the extract is prepared by a process comprising washing the acetonitrile extract with hexane.

53. The method of embodiment 52, wherein the extract is prepared by a process comprising washing the acetonitrile extract twice with hexane.

54. The method of any one of embodiments 46 to 53, wherein the extract is prepared by a process that comprises removing the organic solvent from the acetonitrile extract and redissolving the acetonitrile extract in methanol, dimethyl sulfoxide, or a combination thereof.

55. The method of any one of embodiments 14 to 38, wherein the organic solvent is hexane and the process further comprises subjecting the organic solvent extract to a solvent extraction using a polar organic solvent, optionally wherein the polar organic solvent is acetonitrile.

56. The method of embodiment 55, wherein the process further comprises removing the polar organic solvent from the organic solvent extract and redissolving the organic solvent extract in a second organic solvent.

57. The method of embodiment 56, wherein the second organic solvent comprises methanol, dimethyl sulfoxide, or a combination thereof.

58. The method of any one of embodiments 55 to 57, wherein the process further comprises recovering the organic solvent extract.

59. The method of any one of embodiments 55 to 58, which further comprises preparing the extract.

60. The method of embodiment 59, wherein the extract is prepared by a process comprising the steps described in any one of embodiments 55 to 58.

61. The method of any one of embodiments 1 to 60, wherein the teleost embryo is an eleutheroembryo.

62. The method of any one of embodiments 1 to 61, wherein the teleost embryo is a medaka embryo, a zebrafish embryo or a fathead minnow embryo.

63. The method of embodiment 62, wherein the teleost embryo is a transgenic medaka embryo or a transgenic zebrafish embryo.

64. The method of any one of embodiments 1 to 63, wherein the toxicity effect comprises an acute effect.

65. The method of embodiment 64, in which the acute effect comprises mortality, malformation or a combination thereof.

66. The method of any one of embodiments 1 to 63, in which the toxicity effect comprises a specific effect.

67. The method of embodiment 66, wherein the specific effect is an endocrine activity disruption.

68. The method of embodiment 67, wherein the endocrine activity disruption is estrogen activity disruption, androgen activity disruption, or thyroid activity disruption.

69. The method of embodiment 67 or embodiment 68, wherein the teleost embryo is a transgenic teleost embryo comprising a glycoprotein subunit α (gsuα) promoter operably linked to a marker gene, and optionally wherein determining whether the extract exerts a toxicity effect on the embryo comprises detecting or measuring changes in expression of the marker gene.

70. The method of embodiment 68, wherein the endocrine activity disruption is estrogen activity disruption, wherein the teleost embryo is a transgenic teleost embryo comprising an estrogen sensitive promoter operably linked to a marker gene, and optionally wherein determining whether the extract exerts a toxicity effect on the embryo comprises detecting or measuring changes in expression of the marker gene.

71. The method of embodiment 70, wherein the estrogen sensitive promoter is an aromatase B promoter, and optionally wherein the teleost embryo is a zebrafish embryo or a medaka embryo.

72. The method of embodiment 70, wherein the estrogen sensitive promoter is a choriogenin promotor which is optionally a choriogenin H promoter or a choriogenin L promoter, and optionally wherein the teleost embryo is a zebrafish embryo or a medaka embryo.

73. The method of embodiment 70, wherein the estrogen sensitive promoter is a vitellogenin promoter, and optionally wherein the teleost embryo is a zebrafish embryo or a medaka embryo.

74. The method of embodiment 68, wherein the endocrine activity disruption is androgen activity disruption, wherein the teleost embryo is a transgenic teleost embryo comprising an androgen sensitive promoter operably linked to a marker gene, and optionally wherein determining whether the extract exerts a toxicity effect on the embryo comprises detecting or measuring changes in expression of the marker gene.

75. The method of embodiment 74, wherein the androgen sensitive promoter is a spiggin promoter, and optionally wherein the teleost embryo is a medaka embryo or a zebrafish embryo.

76. The method of embodiment 68, wherein the endocrine activity disruption is thyroid activity disruption, wherein the teleost embryo is a transgenic teleost embryo comprising a thyroid hormone (TH) sensitive promoter operably linked to a marker gene, and optionally wherein determining whether the extract exerts a toxicity effect on the embryo comprises detecting or measuring changes in expression of the marker gene.

77. The method of embodiment 76, wherein the TH sensitive promoter is a thyroid-stimulating hormone subunit β (TSHβ) promoter and optionally wherein the teleost embryo is a medaka embryo or a zebrafish embryo.

78. The method of embodiment 66, wherein the specific effect is a xenobiotic effect, wherein the teleost embryo is a transgenic teleost embryo comprising a xenobiotic sensitive promoter operably linked to a marker gene, and optionally wherein determining whether the extract exerts a toxicity effect on the embryo comprises detecting or measuring changes in expression of the marker gene.

79. The method of embodiment 78, wherein the xenobiotic sensitive promoter is a P450 1A promoter, and optionally wherein the teleost embryo is a medaka embryo or a zebrafish embryo.

80. The method of embodiment 66, wherein the specific effect is a xenobiotic effect, and wherein determining whether the sample or the extract exerts a toxicity effect on the embryo comprises detecting or measuring changes in ethoxyresorufin-O-deethylase (EROD) activity.

81. The method of embodiment 66, wherein the specific effect is a cardiotoxicity effect, and optionally wherein determining whether the extract exerts a toxicity effect on the embryo comprises detecting or measuring alterations in cardiac development and/or blood circulation rate.

82. The method of embodiment 81, wherein the embryo harbors a BMP4 promoter operably linked to a marker gene and wherein detecting or measuring alterations in cardiac development comprises monitoring marker gene expression.

83. The method of embodiment 81, wherein the embryo harbors a gata1 promoter operably linked to a marker gene and wherein detecting or measuring alterations in blood circulation rate comprises monitoring marker gene expression.

84. The method of embodiment 66, wherein the specific effect is a hepatotoxicity effect, and optionally wherein determining whether the extract exerts a toxicity effect on the embryo comprises detecting or measuring changes in liver development.

85. The method of any one of embodiments 69 to 79, 82 and 83, wherein the promoter is native to the teleost embryo.

86. The method of any one of embodiments 69 to 79, 82 and 83, wherein the promoter is not native to the teleost embryo.

87. The method of embodiment 86, wherein the teleost embryo is a zebrafish embryo and the promoter is native to a medaka fish.

88. The method of embodiment 87, wherein the promoter is native to Oryzias melastigma or Oryzias latipes.

89. The method of embodiment 86, wherein the teleost embryo is a medaka embryo and the promoter is native to a zebrafish.

90. The method of embodiment 89, wherein the medaka embryo is an Oryzias melastigma embryo or Oryzias latipes embryo.

91. The method of any one of embodiments 69 to 79, 82, 83 or 85 to 90, wherein the marker gene encodes a fluorescent protein.

92. The method of embodiment 91, wherein the fluorescent protein is a green fluorescent protein (GFP), cyan fluorescent protein (CFP), yellow fluorescent protein (YFP), red fluorescent protein (dsRFP), luciferase (Luc), chloramphenicol acetyltransferase (CAT), 13-galactosidase (LacZ) or β-glucuronidase (Gus).

93. The method of any one of embodiments 69 to 79, 82, 83 or 85 to 90, wherein marker gene encodes an enzyme detectable in a colorimetric assay.

94. The method of embodiment 93, wherein the enzyme is a luciferase, horseradish peroxidase, β-galactosidase, β-glucuronidase, alkaline phosphatase, chloramphenicol acetyl transferase, or alcohol dehydrogenase.

95. The method of any one of embodiments 1 to 94 which is performed in a multiwell plate, optionally a 24-well plate, a 96-well plate or a 384-well plate.

96. The method of any one of embodiments 1 to 95 in which more than one consumable product sample is assayed.

97. The method of embodiment 96 in which each sample is assayed in duplicate or in triplicate.

98. The method of any one of embodiments 1 to 97, which comprises assaying multiple dilutions of a consumable product extract.

99. A method of preparing an extract from a consumable product for toxicant testing, comprising subjecting the consumable product to the process described in any one of embodiments 14 to 43 or 55 to 58.

100. The method of embodiment 99, in which the extract is an acetonitrile extract and step (a) of the process comprises combining the sample with acetonitrile.

101. The method of embodiment 100, in which step (a) of the process comprises combining the sample with acetonitrile and sodium chloride.

102. The method of embodiment 100, in which the process comprises combining the mixture with sodium chloride prior to step (b) of the process.

103. The method of any one of embodiments 100 to 102, in which the process comprises mixing the mixture before step (b).

104. The method of any one of embodiments 100 to 103, in which the process comprises adding anhydrous sodium sulfate to the phase containing the acetonitrile from step (b) to form a second mixture.

105. The method of embodiment 104, in which the process comprises a step of mixing the second mixture and separating a phase containing the acetonitrile from the second mixture.

106. The method of any one of embodiments 100 to 105, in which the process comprises washing the acetonitrile extract with hexane.

107. The method of embodiment 106, in which the process comprises washing the acetonitrile extract twice with hexane.

108. The method of any one of embodiments 100 to 107, in which the process comprises removing the acetonitrile from the acetonitrile extract and redissolving the acetonitrile extract in methanol, dimethyl sulfoxide, or a combination thereof.

While various specific embodiments have been illustrated and described, it will be appreciated that various changes can be made without departing from the spirit and scope of the disclosure(s).

### 1. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A method of determining an overall toxicity in a consumable product, comprising:
a) combining a polar organic solvent and the consumable product to obtain a polar organic extract with an overall toxicity;
b) contacting a teleost embryo with the organic extract of a); and
c) determining whether the extract exerts a toxicity effect on the embryo;
wherein a toxicity effect on the embryo shows an overall toxicity of the consumable product.

2. The method of claim 1, wherein the consumable product is a food, edible oil or feed.

3. The method of claim 1, wherein the food is a packaged food, a dairy product, meat, infant formula or a beverage.

4. The method of claim 1, wherein the polar organic solvent is acetonitrile, methanol, ethanol, propanol, isopropanol, acetone, ethyl acetate, propanol, isopropanol, and a mixture containing two or more solvents thereof.

5. The method of claim 1, wherein the polar organic solvent is acetonitrile.

6. The method of claim 1, wherein the volume of the polar organic solvent combined with the consumable sample to form a mixture is about 1 to about 5 times the volume of the sample, when the sample is a liquid; or the volume of the polar organic solvent combined with the consumable sample to form a mixture is about 1 to about 5 times the weight of the sample, when the sample is a solid or semi-solid.

7. The method of claim 1, wherein the volume of the polar organic solvent combined with the consumable sample to form a mixture is about 1 to about 3 times the volume of the sample, when the sample is a liquid; or the volume of the polar organic solvent combined with the consumable sample to form a mixture is about 1 to about 3 times the weight of the sample, when the sample is a solid or semi-solid.

8. The method of claim 1, wherein before the above step a), water is added to the consumable product when the product is water unsaturated.

9. The method of claim 1, wherein the polar organic extract is further delipidated with a non-polar solvent for a lipid-containing consumable product.

10. The method of claim 9, wherein the lipid-containing consumable product contains a lipid that causes the polar organic extract cannot be completely dried.

11. The method of claim 9, wherein the lipid-containing consumable product contains a lipid higher than 5% (w/w).

12. The method of claim 9, wherein the non-polar solvent is pentane, cyclopentane, hexane, cyclohexane, benzene, toluene, chloroform, diethyl ether, dichloromethane or a mixture containing two or more the solvents thereof.

13. The method of claim 1, wherein in step a), a first salt and/or a sugar can be further added to obtain the polar organic solvent.

14. The method of claim 13, wherein the first salt is sodium chloride, magnesium sulfate, sodium sulfate, calcium sulfate, calcium chloride, magnesium chloride, sodium acetate, ammonium acetate, anhydrous sodium sulfate, anhydrous magnesium sulfate, anhydrous sodium sulfate, anhydrous calcium sulfate, anhydrous calcium chloride, anhydrous calcium sulfate, or a combination thereof.

15. The method of claim 13, wherein the sugar is sucrose.

16. The method of claim 1, wherein the method further comprises a step of adding a second salt to the organic solvent extract from step (a) to form a mixture.

17. The method of claim 16, wherein the second salt is an anhydrous sodium sulfate, anhydrous magnesium sulfate, anhydrous calcium sulfate, anhydrous calcium chloride, or a combination thereof.

18. The method of claim 1, wherein the process further comprises a step of homogenizing the sample prior to step (a); drying the organic solvent extract and redissolving the organic solvent extract in a second organic solvent; or recovering the organic solvent extract.

19. The method of claim 1, wherein the teleost embryo is a zebrafish or medaka or zebrafish embryo.

20. The method of claim 1, wherein the chemical contaminants of the consumable product are determined by a chemical analysis method to establish the correlation between the teleost embryo toxicity test and chemical contaminants.
